(19) United States
(12) Patent Application Publication
(10) Pub. No.: US 2012/0021469 A1
(43) Pub. Date: Jan. 26, 2012

(54) AMIDASES AND METHODS OF THEIR USE

(75) Inventors: Owen Gooding, San Jose, CA (US); Robert J. Jones, Millbrae, CA (US); Gjalt Huisman, San Carlos, CA (US); Jie Yang, Foster City, CA (US); Louis Clark, San Francisco, CA (US)

(73) Assignee: CODEXIS, INC., Redwood City, CA (US)

(21) Appl. No.: 13/256,581

(22) PCT Filed: Mar. 26, 2010
(86) PCT No.: PCT/US10/28874

§ 371 Date: Oct. 13, 2011

Related U.S. Application Data
(60) Provisional application No. 61/164,383, filed on Mar. 27, 2009.

(51) Int. Cl. C12N 9/80; C12N 15/70; C07C 237/10; C12P 13/02; C12P 17/10; C07C 229/26; C12N 15/55; C12N 1/21

(52) U.S. Cl. 435/121; 435/228; 536/23.2; 435/320.1; 435/252.33; 435/129; 562/561; 564/198

(57) ABSTRACT

The disclosure relates to engineered amidase polypeptides and processes of using the polypeptides for chiral resolution of amino acid amide compounds. The disclosure further relates to the polynucleotides that encode the engineered amidase polypeptides and related vectors, host cells, and methods for making the engineered amidase polypeptides.

This application claims the benefit, pursuant 35 U.S.C. §119(e), of U.S. Ser. No. 61/164,383, filed Mar. 27, 2009, which is incorporated herein by reference.

### 1. TECHNICAL FIELD

The present disclosure relates to engineered amidase polypeptides and processes of using the polypeptides for resolution of chiral amino acid amide compounds.

### 2. REFERENCE TO SEQUENCE LISTING, TABLE OR COMPUTER PROGRAM

The “Sequence Listing” submitted electronically concurrently herewith pursuant 37 C.F.R. §1.821 in a computer readable form (CRF) via EFS-Web as file name CX2-011WO1_ST25.txt is incorporated herein by reference. The electronic copy of the Sequence Listing was created on Mar. 19, 2010, and the size on disk is 132 Kbytes.

### 3. BACKGROUND

Levetiracetam is an anti-convulsant therapeutic useful for the treatment of epilepsy and other neurological disorders. It is the chiral form of etiracetam, 2-(2-oxopyrrolidin-1-yl)-butanamide and displays significantly higher protective activity against hypoxia and ischemia than the racemate etiracetam. Levetiracetam possesses several properties that distinguish it from classical anti-epileptic drugs. Levetiracetam does not appear to show significant efficacy in seizure models, such as pentyenetetrazol induced seizures, used to screen for other antiepileptic drugs (Klitgard et al., 1998, Eur. J. Pharmacol. 353:191-206). The drug also does not affect the known biological process affected by other anti-epileptic drugs, such as facilitation of γ-aminobutyric acid (GABA) mediated neurotransmission, activity dependent block of voltage gated sodium channels, inhibition of calcium currents, and inhibition of receptors for excitatory amino acids (e.g., glutamate receptors).

Studies suggest that the cellular target for levetiracetam is SV2A, a protein with specific expression in neurons and endocrine cells (Lynch et al., 2004, Proc. Natl. Acad. Sci. USA 101:9861-9866). The SV2 protein contains 12 transmembrane domains and an amino terminal half having significant amino acid sequence homology to a family of bacterial proteins that transport sugars, citrate, and drugs (Bajjalieh et al., 1992, Science 257(5074):1271-3). SV2-like immunoreactivity is distributed in a reticular and punctate pattern, suggesting its presence in intracellular membranes. Its localization to vesicles, predicted membrane topology, and sequence identity to known transporters suggest that SV2A is a synaptic vesicle-specific transporter. These characteristics suggest that SV2A acts as a modulator of vesicle fusion, although SV2A may have other functions at the presynaptic terminal. The binding of levetiracetam to SV2A proteins suggest that the drug has a biochemical mode of action that is distinct from that of other antiepileptic drugs. Seletracetam and brivaracetam are derivatives of levetiracetam that are substituted at the 4-position on the 2-pyrrolidinone ring (Kenda et al., 2004, J Med. Chem. 47(3):530-49). Both have 10-fold greater affinity for SV2A than does levetiracetam, but brivaracetam may also have modest sodium-channel blocking activity. These derivatives also bind to the SV2A protein.

Various chemical methods have been described for the synthesis of levetiracetam. Resolution of the levetiracetam from the racemic mixture involves separation of the chiral compounds by simulated moving bed (see, e.g., U.S. Pat. Nos. 6,124,473 and 6,107,492). However, as with many chemical processes requiring separation of chiral compounds, the chiral phase is associated with high cost and requires use of large amounts of solvents. Thus, it is desirable to identify alternative methods of generating levetiracetam and various analogs thereof, such as brivaracetam and seletracetam.

### 4. SUMMARY

The present disclosure provides engineered amidase polypeptides, polynucleotides encoding the polypeptides, and methods of using amidase polypeptides for the stereospecific conversion of an (R)-amino acid amide of structural formula (I) to an (R)-amino acid of structural formula (II), thereby allowing the resolution of an (S)-amino acid amide of structural formula (III) from a racemic amino acid amide mixture represented by formula (IV) (wherein R₁ of the structural formulas below represents a lower alkyl of C₁-C₄).

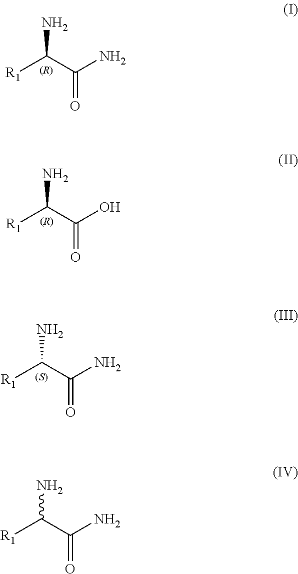

In some embodiments, the engineered amidase polypeptides are capable of stereospecifically converting a racemic mixture of 2-aminobutyramide (VIII) to (R)-2-aminobutyric acid (VI), thereby forming (S)-2-aminobutyramide (VII) in stereomeric excess (i.e., via the stereospecific conversion of (R)-2-aminobutyramide (V) to (R)-2-aminobutyric acid (VI)).

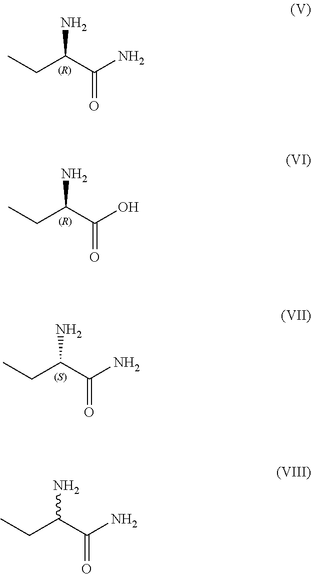

In one aspect, the engineered amidase polypeptides of the disclosure have improved enzyme properties as compared to the naturally occurring amidase of Ochrobactrum anthropi, the sequence of which is represented by SEQ ID NO: 2. In some embodiments, the amidase polypeptides are improved as compared to another engineered amidase polypeptide, such as the polypeptide of SEQ ID NO:4. The improvements in enzyme properties include improvements in, among others, enzyme activity (e.g., conversion rate), pH stability, and reduced byproduct formation. In some embodiments, the amidase polypeptides are characterized by a combination of improved properties, such as increased enzymatic activity, pH stability, and reduced byproduct formation. In some embodiments, the engineered amidase polypeptides are capable of stereospecifically converting (R)-2-aminobutyramide to (R)-aminobutyrate in a racemic mixture of 2-aminobutyramide to form a stereomeric excess of at least 99% of (S)-2-aminobutyramide.

In some embodiments, the amidase polypeptides are improved with respect to enzyme activity as compared to the activity of the amidase polypeptide of SEQ ID NO:2 or SEQ ID NO:4 in converting (R)-2-aminobutyramide to (R)-2-aminobutyric acid. In some embodiments, the engineered amidase polypeptides have at least 1.2 times the enzymatic activity as compared to the activity of SEQ ID NO: 2 at a reaction condition of pH of about 7.5 and a temperature of about 35° C. In some embodiments, the engineered amidase polypeptides have at least 1.5 times the enzymatic activity as compared to the activity of SEQ ID NO: 2 at a reaction condition of pH of about 9.5 and a temperature of about 25° C.

In some embodiments, the improved enzymatic activity of the amidase polypeptides can be characterized by an increase in the conversion rate of the substrate, such as conversion of (R)-2-aminobutyramide in a racemic mixture of 2-aminobutyramide to the corresponding (R)-2-aminobutyrate under a defined condition. In some embodiments, the improved amidase polypeptides are capable of converting the (R)-2-aminobutyramide in a racemic mixture of 2-aminobutyramide to the (R)-2-aminobutyrate at a yield of at least 40%, 45%, 46%, 47%, 48%, 49% or more up to the theoretical yield of 50% of the racemic mixture under the defined condition. In some embodiments, the defined condition is a substrate loading of at least about 300 g/L or at least about 475 g/L of 2-aminobutyramide in its acetate salt form with 1 g/L of amidase polypeptide under reaction conditions of pH of about 7.5 and about 35° C. in a reaction time of about 8 hrs. In some embodiments, the defined condition is a substrate loading of about 300 g/L 2-aminobutyramide and an alkaline reaction condition comprising a pH of about 9.5, temperature of about 25° C., about 5% isopropanol in a reaction time of about 20 hrs.

In some embodiments, the engineered amidase polypeptides are improved with respect to the level of byproducts formed in the conversion of 2-aminobutyramide to (R)-2-aminobutyric acid. In some embodiments, the level of byproduct is less than 10%, less than 7.5%, less than 6%, less than 5%, less than 4%, less than 3%, less than 2%, less than 1%, less than 0.5%, less than 0.2%, or less than 0.1% of the total products (i.e., all products derivatized with amine reacting agent o-phthalaldehyde (OPA), including 2-aminobutyramide and 2-aminobutyric acid), as determined by fluorometry of products following derivatization with OPA.

In some embodiments, the improved amidase polypeptides herein can comprise an amino acid sequence that has at least about 80%, 85%, 86%, 87%, 88%, 89%, 90%, 91%, 92%, 93%, 94%, 95%, 96%, 97%, 98%, 99% or more identity as compared to a reference sequence of SEQ ID NO:2 or an engineered amidase polypeptide of SEQ ID NO:4 and having one or more of the improved properties.

In some embodiments, the improved amidase polypeptide comprises an amino acid sequence that has one or more residue differences as compared to the polypeptide of SEQ ID NO:2 at the following residue positions: X38; X149; X175; X278; X290; X291; X315; X317; X353; X363; X367; X376; X405; X414; X516; and X518. Guidance for the amino acid residues that can be selected for the specified residue positions are described in the detailed description.

In some embodiments, the improved amidase polypeptide comprises an amino acid sequence that includes at least one or more of the following features: residue corresponding to X290 is a polar or acidic residue; residue corresponding to X291 is non-polar residue; residue corresponding to X317 is an acidic residue; residue corresponding to X367 is an acidic residue; and residue corresponding to X414 is a polar residue.

In some embodiments, the improved amidase polypeptide comprises an amino acid sequence that includes at least the following features: residue corresponding to X290 a polar or acidic residue, particularly serine, threonine, glutamine or glutamic acid.

In some embodiments, the improved amidase polypeptide comprises an amino acid sequence that includes at least the following features: residue corresponding to X291 is a non-polar or aliphatic residue, particularly methionine.

In some embodiments, the improved amidase polypeptide comprises an amino acid sequence that includes at least the following features: residue corresponding to X317 is an acidic residue, particularly aspartic acid; residue corresponding to X367 is an acidic residue, particularly aspartic acid; and residue corresponding to X414 is a polar residue, particularly asparagine.

In some embodiments, the improved amidase polypeptide comprises an amino acid sequence that includes at least the following features: residue corresponding to X290 is a polar or acidic residue, particularly serine, threonine, glutamine or glutamic acid; residue corresponding to X317 is an acidic residue, particularly aspartic acid; residue corresponding to X367 is an acidic residue, particularly aspartic acid; and residue corresponding to X414 is a polar residue, particularly asparagine.

In some embodiments, the improved amidase polypeptides comprising an amino acid sequence having the specified features or combination of features for residues X290, X291, X317, X367, and/or X414 as provided in the foregoing and descriptions herein, can further include one or more residue differences as compared to SEQ ID NO:2 at other residue positions. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other amino acid residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other amino acid residue positions. In some embodiments, the residue differences at other residue positions comprise conservative mutations. In some embodiments, an engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% or more identical to a reference amino acid sequence based on SEQ ID NO:2 having the features or combinations of features described herein for the residues corresponding to X290, X291, X317, X367, and/or X414, with the proviso that the engineered amidase polypeptides have at the residues corresponding to X290, X291, X317, X367, and/or X414 at least the features provided herein.

In some embodiments, the amidase polypeptide capable of stereospecifically converting (R)-2-aminobutyramide to (R)-2-aminobutyrate in a stereomeric excess of at least 99% of (S)-2-aminobutyramide comprises an amino acid sequence selected from SEQ ID NO: 6, 8, 10, 12, 14, 16, 18, 20, and 22.

In some embodiments, the improved amidase polypeptide is capable of stereospecifically converting at least 40%, 45%, 46%, 47%, 48% or 49% of 475 g/L of a racemic mixture of 2-aminobutyramide to (R)-2-aminobutyrate at a reaction condition of a pH of about 7.5 and about 35° C. in about 8 hrs in presence of about 1 g/L of amidase polypeptide. In some embodiments, the improved amidase polypeptide capable of stereospecifically converting at least 40%, 45%, 46%, 47%, 48% or 49% of 475 g/L of a racemic mixture of 2-aminobutyramide to (R)-2-aminobutyrate under reaction conditions of pH 7.5 and 35° C. in about 8 hrs comprises an amino acid sequence selected from SEQ ID NO: 6, 8, 10, 12, 14, 16, 18, 20, and 22.

In some embodiments, the improved amidase polypeptide is capable of stereospecifically converting at least 40%, 45%, 46%, 47%, 48% or 49% of 300 g/L of a racemic mixture of 2-aminobutyramide acetate salt to (R)-2-aminobutyrate acetate salt under reaction conditions of pH 9.5 and 25° C. in about 20 hrs. In some embodiments, the improved amidase polypeptide capable of converting at least 40%, 45%, 46%, 47%, 48% or 49% of 300 g/L of a racemic mixture of 2-aminobutyramide acetate salt to (R)-2-aminobutyrate acetate salt under reaction conditions of pH 9.5 and 25° C. in about 20 hrs comprises an amino acid sequence selected from SEQ ID NO: 6, 8, 10, 12, 14, 16, 18, 20, and 22.

In another aspect, the present disclosure provides polynucleotides encoding the engineered amidases described herein or polynucleotides that hybridize to such polynucleotides under highly stringent conditions. The polynucleotide can include promoters and other regulatory elements useful for expression of the encoded engineered amidase, and can utilize codons optimized for specific desired expression systems. Exemplary polynucleotides include, but are not limited to, SEQ ID NO: 5, 7, 9, 11, 13, 15, 17, 19, or 21.

In another aspect, the present disclosure provides host cells comprising the polynucleotides and/or expression vectors described herein. The host cells may be E. coli, or they may be a different organism. The host cells can be used for the expression and isolation of the engineered amidase enzymes described herein, or, alternatively, they can be used directly for the conversion of the substrate to the stereoisomeric product.

In a further aspect, the present disclosure provides methods of using amidases for stereospecifically converting a racemic mixture of the amino acid amide of formula (IV) to the (R)-amino acid (II), wherein R₁ is a (C₁-C₄) alkyl, thereby allowing chiral resolution of the (S)-amino acid amide of formula (III) from the (R)-amino acid amide (I), as illustrated in Scheme 1:

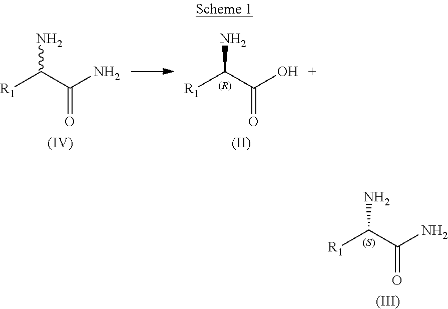

Accordingly, in some embodiments, the method can comprise contacting a racemic substrate mixture of formula (IV) (which is a mixture of compounds (I) and (III) with an amidase under reaction conditions suitable to form a mixture of the compound of formula (II) and compound of formula (III), thereby resulting in a stereomeric excess the compound of formula (III) relative to its opposite enantiomer the compound of formula (I).

In some embodiments, the method can comprise contacting a mixture, such as a racemic mixture of formula (VIII), which comprises (R)-2-aminobutyramide (V) and (S)-2-aminobutyramide (VII), with a polypeptide having amidase activity thereby converting the (R)-2-aminobutyramide to the corresponding (R)-2-aminobutyric acid (VI) for chiral resolution of the (S)-aminobutyramide (VII) from the (R)-2-aminobutyramide (V), as illustrated in Scheme 2:

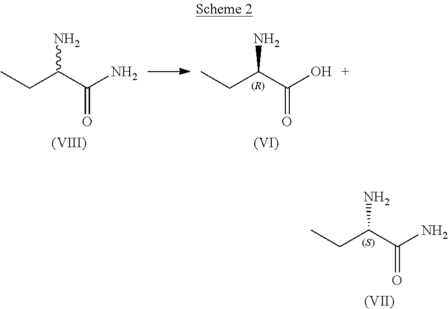

In some embodiments, the method comprises contacting a substrate that is a racemic mixture of (R)-2-aminobutyramide (V) and (S)-2-aminobutyramide (VII) with an amidase polypeptide to convert the (R)-2-aminobutyramide to the corresponding (R)-2-aminobutyric acid (VI) under reaction conditions suitable to provide (S)-2-aminobutyramide (VII) in at least 99%, 99.1%, 99.2%, 99.3%, 99.4%, 99.5%, 99.6%, 99.7%, 99.8%, or 99.9% stereomeric excess relative to (R)-2-aminobutyramide.

In some embodiments, the method comprises contacting a substrate that is a racemic mixture of (R)-2-aminobutyramide and (S)-2-aminobutyramide with an amidase polypeptide under reaction conditions of pH of about 7.5, a temperature of about 35° C., about 475 g/L, or even higher loading of 2-aminobutyramide substrate, and about 1 g/L of engineered amidase polypeptide, thereby converting at least 40%, 45%, 46%, 47%, 48% or 49% of substrate to (R) 2-aminobutyrate in a reaction time of about 8 hrs.

In some embodiments, the method comprises contacting a substrate that is a racemic mixture of (R)-2-aminobutyramide and (S)-2-aminobutyramide with an amidase polypeptide under reaction conditions of about pH 9.5, a temperature of about 25° C., about 300 g/L of 2-aminobutyramide substrate, thereby converting at least 40%, 45%, 46%, 47%, 48% or 49% of the substrate to (R)-2-aminobutyrate in a reaction time of about 20 hrs.

Because the amidase mediated reaction is shown herein to have greater efficiency using a lower carboxylic acid salt or sulfate salt of the amino acid amides, further provided herein are compositions comprising lower carboxylic acid salts or sulfate salts of the (R)- and (S)-amino acid amides or mixtures thereof, as illustrated below by compounds of formula (Ia) and formula (IIIa), and racemic mixture represented by formula (IVa), where R₁ can be a lower alkyl, i.e., (C₁-C₄)alkyl.

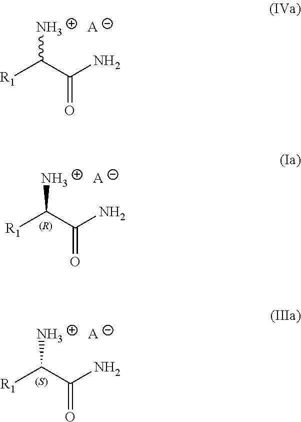

In some embodiments, the lower carboxylic acid salts or sulfate salts are formate, acetate, propionate, or sulfate salts of the amino acid amides. In some embodiments, the compositions comprise formate, acetate, or propionate salts of a racemic mixture of amino acid amides as represented by the structural formula (IVa).

In some embodiments, useful for the methods herein are compositions of lower carboxylic acid salts or sulfate salts of (R)-2-aminobutyramide (V) or (S)-2-aminobutyramide (VII), or mixtures thereof, such as salts of a racemic mixture (VII) of (R)-2-aminobutyramide (V) and (S)-2-aminobutyramide (VII). In some embodiments, the salts of a mixture of (R)-2-aminobutyramide and (S)-2-aminobutyramide are formate, acetate, propionate or sulfate salts of the 2-aminobutyramide.

In some embodiments, the method for stereospecifically converting racemic 2-aminobutyramide (VIII) to (R)-2-aminobutyrate (VI) comprises contacting or incubating a lower carboxylic acid salt or sulfate salt of the racemic 2-aminobutyramide with an amidase under reaction conditions suitable for converting (R)-2-aminobutyramide to (R)-2-aminobutyrate, thereby forming (S)-2-aminobutyramide (salt thereof) in stereomeric excess. In some embodiments, the amidase suitable for use in the method can be a naturally occurring amidase, including, by way of example and not limitation, any of the following amidase polypeptides: S02709264 (SEQ ID NO: 2); gi|75475218|sp|Q9ZBA9.3|DAP_OC (SEQ ID NO: 23); gi|153008743|ref|YP_{—}001369958 (SEQ ID NO: 24); gi|23500673|ref|NP_{—}700113.1| (SEQ ID NO: 25); gi|17988695|ref|NP_{—}541328.1| (SEQ ID NO: 26); gi|148558345|ref|YP_{—}001257867 (SEQ ID NO: 27); gi|126464718|ref|YP_{—}001045831 (SEQ ID NO: 28); gi|77465254|ref|YP_{—}354757.1| (SEQ ID NO: 29); gi|169785843|ref|XP_{—}001827382 (SEQ ID NO: 30); gi|58039691|ref|YP_{—}191655.1 (SEQ ID NO: 31); gi|114762789|ref|ZP_{—}01442223.1 (SEQ ID NO: 32); gi|46126321|ref|XP_{—}387714.1| (SEQ ID NO: 33); gi|76057819|emb|CAH19237.1|_p (SEQ ID NO: 34); and gi|145241538|ref|XP_{—}001393415 (SEQ ID NO: 35).

In some embodiments, the amidase polypeptide can comprise an engineered amidase described herein, including, among others, the polypeptide comprising the amino acid sequence selected from SEQ ID NO: 6, 8, 10, 12, 14, 16, 18, 20, and 22.

In some embodiments, the (S)-2-aminobutyramide can be used for preparing levetiracetam, (2S)-2-(2-oxopyrrolidin-1-yl)butanamide, having the following structural formula (X):

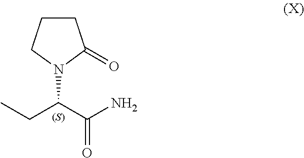

Accordingly, in a method for the synthesis of levetiracetam, a step in the method can comprise contacting a mixture of (R)-2-aminobutyramide and (S)-2-aminobutyramide, such as in the form of lower carboxylic acid salts or sulfate salts, with an amidase to form (S)-2-aminobutyramide in enantiomeric excess of the (R)-2-aminobutyramide.

In some embodiments, amidase polypeptides can be used in a method for preparing brivaracetam, (2S)-2-[(4R)-2-oxo-4-propylpyrrolidin-1-yl]butanamide, having the following structural formula (XI):

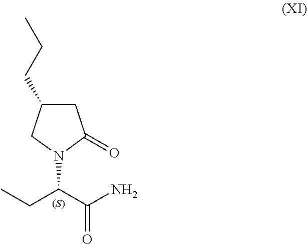

Thus, in some embodiments, in a method for the synthesis of brivaracetam, a step in the method can comprise contacting a mixture of (R)-2-aminobutyramide and (S)-2-aminobutyramide, such as in the form of lower carboxylic acid salts or sulfate salts, with an amidase to form (S)-2-aminobutyramide in enantiomeric excess of the (R)-2-aminobutyramide.

In some embodiments, amidase polypeptides can be used in a method for preparing seletracetam, (2S)-2-[(4R)-4-(2,2-difluoroethenyl)-2-oxo-pyrrolidin-1-yl]butanamide, having the following structural formula (XII):

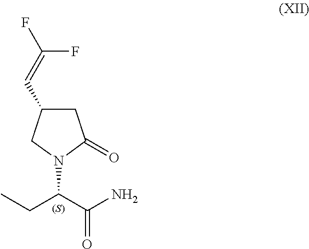

Thus, in some embodiments, in a method for the synthesis of seletracetam, a step in the method can comprise contacting a mixture of (R)-2-aminobutyramide and (S)-2-aminobutyramide, such as in the form of lower carboxylic acid salts or sulfate salts, with an amidase to form (S)-2-aminobutyramide in enantiomeric excess of the (R)-2-aminobutyramide.

In some embodiments, in the processes for synthesis of levetiracetam, brivaracetam, or seletracetam of this disclosure, the process can further comprise isolating the (S)-2-aminobutyramide, wherein the (S)-2-aminobutyramide is isolated by extraction with a polar organic solvent. In some embodiments, the polar organic solvent is selected from acetonitrile, methanol, ethanol, butanol, isopropanol, isobutanol, tert-butanol, acetone, and methylethylketone. In some embodiments, the extraction method is selected from a step-wise or a continuous extraction. In some embodiments, the processes can comprise isolation of a lower carboxylic acid salts or sulfate salt (S)-2-aminobutyramide, wherein the extraction is a step-wise extraction with isopropanol.

### 5. BRIEF DESCRIPTION OF THE FIGURES

FIG. 1 depicts a plot showing the increased pH stability of some of the various engineered amidase polypeptide variants generated by directed evolution relative to the wild-type amidase polypeptide. As shown by the plots, pH stability and percent conversion at alkaline conditions (pH 9.4) with 300 g/L substrate loading can be increased significantly in the variants.

FIG. 2 shows the % conversion to (R)-2-aminobutyric acid and the % byproducts in the reaction mixture carried out by various engineered amidases under a reaction condition of 350 g/L of racemic 2-aminobutyramide, 5% isopropanol, pH 7.5, and temperature of 35° C.

FIG. 3A depicts a chromatogram of a reaction mixture treated with wild-type amidase polypeptide (SEQ ID NO: 2) and separated by HPLC, showing presence of byproducts in the wild-type amidase activity as shown by the “impurity 1” and “impurity 2” peaks. FIG. 3B depicts an HPLC chromatogram of a standard mixture (i.e., untreated with enzyme) containing (R)-2-aminobutyric acid, (R)-2-aminobutyramide, and (S)-2-aminobutyramide.

FIG. 4A and FIG. 4B show HPLC chromatograms of a reaction mixture resulting from reacting racemic 2-aminobutyramide with an engineered amidase polypeptide (SEQ ID NO:10) for 4 hrs or 24 hrs, respectively. As shown by comparison of FIGS. 4A and 4B, the percentage conversion to R-acid product increases and the percentage of byproducts (corresponding to peaks at elution times between 8.5 and 12 min) decreases with the longer incubation time with the engineered enzyme. This further decrease in byproducts likely occurs due to conversion of R-amide substrate dimers.

### 6. DETAILED DESCRIPTION

### Definitions

As used herein, the following terms are intended to have the following meanings.

“Amidase” refers to a polypeptide having an activity capable of hydrolyzing or converting an amino acid amide (e.g., a monocarboxylic acid amide) to its corresponding amino acid (e.g., monocarboxylate) along with formation of NH₃. Amidases can be characterized as an L-amidase or D-amidase based on its stereospecificity for the L-amino acid amides or the D-amino acid amides. In the present disclosure, the amidase polypeptides are capable of stereospecifically hydrolyzing or converting the compound of structural formula (I) to the corresponding product of structural formula (II), thereby resulting in resolution of the compound of formula (III) from the compound of formula (I). Amidases as used herein include naturally occurring (wild-type) amidases as well as non-naturally occurring amidases.

“Engineered amidase polypeptide” as used herein refers to an amidase polypeptide having a variant sequence generated by human manipulation (e.g., a sequence generated by directed evolution of a naturally occurring parent enzyme or of a variant previously derived from a naturally occurring enzyme).

“Byproducts” as used herein refers to a compound or composition formed during the conversion or hydrolysis of an amino acid amide substrate by an amidase other than the amino acid amide substrate (both the (R)- and (S)-enantiomers) and the corresponding amino acid products (both the (R)- and (S)-enantiomers). For example, for the amidase catalyzed conversion of the substrate (R)-2-aminobutyramide in a racemic mixture of 2-aminobutyramide, a byproduct would be a 2-aminobutyramide dimer such as the compound of formula (IX) or any other compound other than (R)-2-aminobutyric acid, (S)-2-aminobutyric acid, (R)-2-aminobutyramide, and (S)-2-aminobutyramide.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

“Polynucleotides” or “oligonucleotides” refer to nucleobase polymers or oligomers in which the nucleobases are connected by sugar phosphate linkages (sugar-phosphate backbone). Nucleobase or base include naturally occurring and synthetic heterocyclic moieties commonly known to those who utilize nucleic acid or polynucleotide technology or utilize polyamide or peptide nucleic acid technology to thereby generate polymers that can hybridize to polynucleotides in a sequence-specific manner. Non-limiting examples of nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Exemplary poly- and oligonucleotides include polymers of 2′ deoxyribonucleotides (DNA) and polymers of ribonucleotides (RNA). A polynucleotide may be composed entirely of ribonucleotides, entirely of 2′ deoxyribonucleotides or combinations thereof.

“Coding sequence” refers to that portion of a nucleic acid (e.g., a gene) that encodes an amino acid sequence of a protein.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. Mol. Biol. 215: 403-410 and Altschul et al., 1977, Nucleic Acids Res. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915).

Other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison Wis.), using default parameters provided.

“Reference sequence” refers to a defined sequence to which an altered sequence is compared. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a comparison window to identify and compare local regions of sequence similarity.

The term “reference sequence” is not intended to be limited to wild-type sequences, and can include engineered or altered sequences. For example, in some embodiments, a “reference sequence” can be a previously engineered or altered amino acid sequence. For instance, a “reference sequence based on SEQ ID NO:2 having a glycine residue at position X315” refers to a reference sequence corresponding to SEQ ID NO:2 with a glycine residue at X315 (whereas the un-altered version of SEQ ID NO:2 has glutamate at X315).

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Substantial identity” refers to a polynucleotide or polypeptide sequence that has at least 80 percent sequence identity, at least 85 percent sequence identity, at least 89 percent sequence identity, at least 95 percent sequence identity, and even at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered amidase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Stereoselectivity” or “stereospecificity” refer to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (e.e.) calculated therefrom according to the formula [major enantiomer−minor enantiomer]/[major enantiomer+minor enantiomer]. Where the stereoisomers are diastereoisomers, the stereoselectivity sometimes is referred to as diastereoselectivity, the fraction (typically reported as a percentage) of one diastereomer in a mixture of two diastereomers, commonly alternatively reported as the diastereomeric excess (d.e.). Enantiomeric excess and diastereomeric excess are types of stereomeric excess.

“Highly stereoselective” or “highly stereospecific” refers to a chemical or enzymatic reaction that is capable of preferentially converting a substrate (e.g., (R)-2-aminobutyramide) to its corresponding product (e.g., (R)-2-aminobutyric acid) with at least 85% stereomeric excess.

“Improved enzyme property” refers to any enzyme property made better or more desirable for a particular purpose as compared to that property found in a reference enzyme. For the engineered amidase polypeptides described herein, the comparison is generally made to the wild-type amidase enzyme, although in some embodiments, the reference amidase can be another improved engineered amidase. Enzyme properties for which improvement is desirable include, but are not limited to, enzymatic activity (which can be expressed in terms of percent conversion of the substrate in a period of time), thermal stability, pH stability or activity profile, cofactor requirements, refractoriness to inhibitors (e.g., product inhibition), stereospecificity, and stereoselectivity (including enantioselectivity).

“Increased enzymatic activity” or “increased activity” or “increased conversion rate” refers to an improved property of an engineered enzyme, which can be represented by an increase in specific activity (e.g., product produced/time/weight protein) or an increase in percent conversion of the substrate to the product (e.g., percent conversion of starting amount of substrate to product in a specified time period using a specified amount of transaminase) as compared to a reference enzyme. Exemplary methods to determine enzyme activity and conversion rate are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 100% improved over the enzymatic activity of the corresponding wild-type amidase enzyme, to as much as 200%, 500%, 1000%, or more over the enzymatic activity of the naturally occurring amidase or another engineered amidase from which the amidase polypeptides were derived. In specific embodiments, the engineered amidase enzyme exhibits improved enzymatic activity in the range of a 100% to 200%, 200% to 1000% or more than a 1500% improvement over that of the parent, wild-type or other reference amidase enzyme. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 10⁸ to 10⁹ (M⁻¹ s⁻¹). Hence, any improvements in the enzyme activity of the amidase will have an upper limit related to the diffusion rate of the substrates acted on by the amidase enzyme. Amidase activity can be measured by any one of standard assays used for measuring amidase, such as the assay condition described in Example 8. Comparisons of enzyme activities or conversion rates are made using a defined preparation of enzyme, a defined assay under a set condition, and one or more defined substrates, as further described in detail herein. Generally, when lysates are compared, the numbers of cells and/or the amount of protein assayed are determined as well as use of identical expression systems and identical host cells to minimize variations in amount of enzyme produced by the host cells and present in the lysates.

“Conversion” refers to the enzymatic transformation of a substrate to the corresponding product. “Percent conversion” refers to the percent of the substrate that is converted to the product within a period of time under specified conditions. Thus, for example, the “activity” or “conversion rate” of an amidase polypeptide can be expressed as “percent conversion” of the substrate to the product.

“Thermostable” or “thermal stable” are used interchangeably to refer to a polypeptide that is resistant to inactivation when exposed to a set of temperature conditions (e.g., 40-80° C.) for a period of time (e.g., 0.5-24 hrs) compared to the untreated enzyme, thus retaining a certain level of residual activity (more than 60% to 80% for example) after exposure to elevated temperatures.

“Solvent stable” refers to a polypeptide that maintains similar activity (more than e.g., 60% to 80%) after exposure to varying concentrations (e.g., 5-99%) of solvent, (e.g., isopropyl alcohol, dimethylsulfoxide, tetrahydrofuran, 2-methyltetrahydrofuran, acetone, toluene, butylacetate, methyl tert-butylether, acetonitrile, etc.) for a period of time (e.g., 0.5-24 hrs) compared to the untreated enzyme.

“pH stable” refers to a polypeptide that maintains similar activity (more than e.g. 60% to 80%) after exposure to high or low pH (e.g. 8 to 12 or 4.5-6) for a period of time (e.g. 0.5-24 hrs) compared to the untreated enzyme.

“Thermo- and solvent stable” refers to a polypeptide that is both thermostable and solvent stable.

“Derived from” as used herein in the context of engineered enzymes identifies the originating enzyme, and/or the gene encoding such enzyme, upon which the engineering was based. For example, the engineered amidase enzyme having variant polypeptide sequence SEQ ID NO: 20 was obtained by artificially mutating, over multiple generations the polynucleotide encoding the wild-type amidase enzyme of SEQ ID NO:2. Thus, this engineered amidase enzyme is “derived from” the wild-type amidase of SEQ ID NO: 2.

“Amino acid” or “residue” as used in context of the polypeptides disclosed herein refers to the specific monomer at a sequence position (e.g., E315 indicates that the “amino acid” or “residue” at position 315 of SEQ ID NO: 2 is a glutamate.)

“Hydrophilic Amino Acid or Residue” refers to an amino acid or residue having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include L-Thr (T), L-Ser (S), L-His (H), L-Glu (E), L-Asn (N), L-Gln (Q), L-Asp (D), L-Lys (K) and L-Arg (R).

“Acidic Amino Acid or Residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic Amino Acid or Residue” refers to a hydrophilic amino acid or residue having a side chain exhibiting a pK value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Polar Amino Acid or Residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S) and L-Thr (T).

“Hydrophobic Amino Acid or Residue” refers to an amino acid or residue having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include L-Pro (P), L-Ile (I), L-Phe (F), L-Val (V), L-Leu (L), L-Trp (W), L-Met (M), L-Ala (A) and L-Tyr (Y).

“Aromatic Amino Acid or Residue” refers to a hydrophilic or hydrophobic amino acid or residue having a side chain that includes at least one aromatic or heteroaromatic ring. Genetically encoded aromatic amino acids include L-Phe (F), L-Tyr (Y) and L-Trp (W). Although owing to the pKa of its heteroaromatic nitrogen atom L-His (H) it is sometimes classified as a basic residue, or as an aromatic residue as its side chain includes a heteroaromatic ring, herein histidine is classified as a hydrophilic residue or as a “constrained residue” (see below).

“Constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry because it also has a five membered ring.

“Non-polar Amino Acid or Residue” refers to a hydrophobic amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M) and L-Ala (A).

“Aliphatic Amino Acid or Residue” refers to a hydrophobic amino acid or residue having an aliphatic hydrocarbon side chain. Genetically encoded aliphatic amino acids include L-Ala (A), L-Val (V), L-Leu (L) and L-Ile (I).

“Cysteine” or amino acid L-Cysteine (C) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges. The ability of L-Cys (C) (and other amino acids with —SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure L-Cys (C) is categorized into its own unique group.

“Small Amino Acid or Residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser (S), L-Thr (T) and L-Asp (D).

“Hydroxyl-containing Amino Acid or Residue” refers to an amino acid containing a hydroxyl (—OH) moiety. Genetically-encoded hydroxyl-containing amino acids include L-Ser (S) L-Thr (T) and L-Tyr (Y).

“Amino acid difference” or “residue difference” refers to a change in the residue at a specified position of a polypeptide sequence when compared to a reference sequence. For example, a residue difference at position E315, where the reference sequence has a glutamate, refers to a change of the residue at position X315 to any residue other than glutamate. As disclosed herein, an engineered amidase enzyme can include one or more residue differences relative to a reference sequence, where multiple residue differences typically are indicated by a list of the specified positions where changes are made relative to the reference sequence (e.g., “one or more residue differences as compared to SEQ ID NO:2 at the following residue positions: X38; X149; X175; X278; X290; X291; X315; X317; X353; X363; X367; X376; X405; X414; X516; and X518.”)

A “conservative” amino acid substitution (or mutation) refers to the substitution of a residue with a residue having a similar side chain, and thus typically involves substitution of the amino acid in the polypeptide with amino acids within the same or similar defined class of amino acids. However, as used herein, in some embodiments, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. Table 1 below shows exemplary conservative substitutions.

TABLE 1
Conservative Substitutions

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P, H | Other constrained (P, H) |
| N, Q, S, T | Other polar (N, Q, S, T) |
| Y, W, F | Other aromatic (Y, W, F) |
| C | None |

“Non-conservative substitution” refers to substitution or mutation of an amino acid in the polypeptide with an amino acid with significantly differing side chain properties. Non-conservative substitutions may use amino acids between, rather than within, the defined groups listed above. In one embodiment, a non-conservative mutation affects (a) the structure of the peptide backbone in the area of the substitution (e.g., proline for glycine) (b) the charge or hydrophobicity, or (c) the bulk of the side chain.

“Deletion” refers to modification to the polypeptide by removal of one or more amino acids from the reference polypeptide. Deletions can comprise removal of 1 or more amino acids, 2 or more amino acids, 3 or more amino acids, 4 or more amino acids, 5 or more amino acids, 6 or more amino acids, 7 or more amino acids, 8 or more amino acids, 10 or more amino acids, 12 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered amidase enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. In some embodiments, the improved engineered amidase enzymes comprise insertions of one or more amino acids to the naturally occurring amidase polypeptide as well as insertions of one or more amino acids to other engineered amidase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Fragment” as used herein refers to a polypeptide that has an amino-terminal and/or carboxy-terminal deletion, but where the remaining amino acid sequence is identical to the corresponding positions in the sequence. Fragments can be at least 14 amino acids long, at least 20 amino acids long, at least 50 amino acids long or longer, and up to 70%, 80%, 90%, 95%, 98%, and 99% of a full-length amidase polypeptide.

“Fusion polypeptide” refers to a polypeptide encoded by a nucleic acid comprising the coding sequence for a first polypeptide and the coding sequence (with or without introns) for a second polypeptide in which the coding sequences are adjacent and in the same reading frame such that, when the fusion construct is transcribed and translated in a host cell, a polypeptide is produced in which the C-terminus of the first polypeptide is joined to the N-terminus of the second polypeptide. A “fusion polypeptide” refers to the polypeptide product of the fusion construct.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The improved amidase enzymes may be present within a cell, present in the cellular medium, or prepared in various forms, such as lysates or isolated preparations. As such, in some embodiments, the improved amidase enzyme can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure amidase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated improved amidase polypeptide is a substantially pure polypeptide composition.

“Stringent hybridization” is used herein to refer to conditions under which nucleic acid hybrids are stable. As known to those of skill in the art, the stability of hybrids is reflected in the melting temperature (Tₘ) of the hybrids. In general, the stability of a hybrid is a function of ionic strength, temperature, G/C content, and the presence of chaotropic agents. The Tₙ, values for polynucleotides can be calculated using known methods for predicting melting temperatures (see, e.g., Baldino et al., Methods Enzymology 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res 18:6409-6412 (erratum, 1991, Nucleic Acids Res 19:698); Sambrook et al., supra); Suggs et al., 1981, In Developmental Biology Using Purified Genes (Brown et al., eds.), pp. 683-693, Academic Press; and Wetmur, 1991, Crit. Rev Biochem Mol Biol 26:227-259. All publications incorporate herein by reference). In some embodiments, the polynucleotide encodes the polypeptide disclosed herein and hybridizes under defined conditions, such as moderately stringent or highly stringent conditions, to the complement of a sequence encoding an engineered amidase enzyme of the present disclosure.

“Hybridization stringency” relates to such washing conditions of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. “High stringency hybridization” refers generally to conditions that are about 10° C. or less from the thermal melting temperature Tₙ, as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. Exemplary high stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Heterologous” polynucleotide refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the amidase polypeptides may be codon optimized for optimal production from the host organism selected for expression.

“Preferred, optimal, high codon usage bias codons” refers interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG Codon Preference, Genetics Computer Group Wisconsin Package; CodonW, John Peden, University of Nottingham; McInerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” refers to all components that are necessary or advantageous for the expression of a polynucleotide and/or polypeptide of interest. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” refers to a configuration in which a control sequence is appropriately placed at a position relative to a polynucleotide sequence (e.g., in a functional relationship) such that the control sequence directs or regulates the expression of a polynucleotide and/or polypeptide.

“Promoter sequence” refers to a nucleic acid sequence that is recognized by a host cell for expression of a polynucleotide of interest (e.g., a coding region). The control sequence may comprise an appropriate promoter sequence. The promoter sequence contains transcriptional control sequences, which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

### Polypeptides and Uses Thereof

The present disclosure provides engineered amidases capable of stereospecifically converting or hydrolyzing an (R) amino acid amide of structural formula (I) to the (R)-2-amino acid of structural formula (II), where R₁ is a lower alkyl, i.e., (C₁-C₄)alkyl:

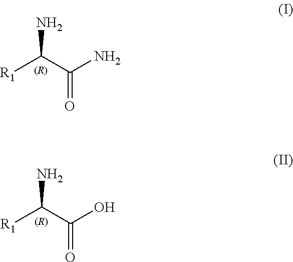

A (C₁-C₄)alkyl encompasses a straight chain or branched non-cyclic hydrocarbon having from 1 to 4 carbon atoms. Representative straight chain (C₁-C₄) alkyls include methyl, ethyl, n-propyl, and n-butyl. Representative branched (C₁-C₄) alkyls include isopropyl, sec-butyl, iso-butyl, and tert-butyl.

In some embodiments, the lower alkyl can be a linear or branched alkyl. Use of the stereospecific amidases with a substrate having a mixture of (R) and (S) amino acid amides, such as a racemic mixture of structure (IV), results in a product having a stereomeric excess of the (S) amino acid amides of structural formula (III) over the (R) amino acid amides of formula (I):

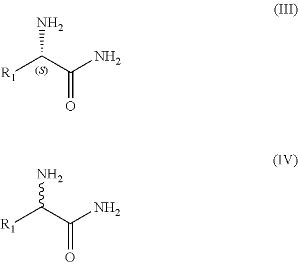

In some embodiments, the stereospecific amidases can be used to convert or hydrolyze (R)-2-aminobutyramide of structural formula (V) to the corresponding (R)-2-aminobutyrate of structural formula (VI):

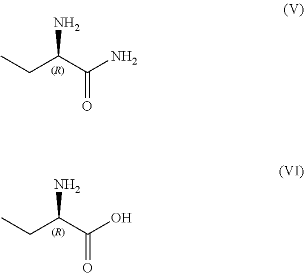

Use of a substrate having a mixture of (R)- and (S)-2-aminobutyramide, such as a racemic mixture of structure (VIII) results in a product having a stereomeric excess of (S)-2-aminobutyramide of formula (VII):

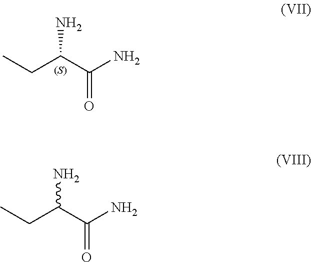

As described herein, the (S)-2-aminobutyramide can be used for the synthesis of levetiracetam, an anti-convulsant used to treat epilepsy.

The amidases of the disclosure are characterized by an improved property as compared to the naturally occurring, wild-type amidase from Ochrobactrum anthropi, represented by SEQ ID NO:2. Enzyme properties for which improvement is desirable include, but are not limited to, enzymatic activity, thermal stability, pH activity/stability profile, refractoriness to inhibitors (e.g., product inhibition), stereospecificity, product purity, and solvent stability. The improvements in the amidase enzyme can relate to a single enzyme property, such as pH stability/activity, or a combination of different enzyme properties, such as enzymatic activity and pH stability.

In some embodiments, for the purposes herein, the engineered amidase with improved enzyme activity can be described with reference to amidase from Ochrobactrum anthropi corresponding to SEQ ID NO:2, or with reference to another engineered amidase, such as SEQ ID NO:4. The amino acid residue position is determined in these amidases beginning from the initiating methionine (M) residue (i.e., M represents residue position 1), although it will be understood by the skilled artisan that this initiating methionine residue may be removed by biological processing machinery, such as in a host cell or in vitro translation system, to generate a mature protein lacking the initiating methionine residue. Therefore, amino acid sequences encoding engineered amidases of the present invention comprise an optional methionine at position 1.

The polypeptide sequence position at which a particular amino acid or amino acid change (“residue difference”) is present is sometimes described herein as “Xn”, or “position n”, where n refers to the residue position with respect to the reference sequence. A specific substitution mutation, which is a replacement of the specific residue in a reference sequence with a different specified residue may be denoted by the conventional notation “X(number)Y”, where X is the single letter identifier of the residue in the reference sequence, “number” is the residue position in the reference sequence (e.g., the wild-type amidase of SEQ ID NO: 2), and Y is the single letter identifier of the residue substitution in the engineered sequence.

In some embodiments, the improved property of the amidase polypeptides is with respect to an increase in enzymatic activity at a reaction condition of pH 7.5 at 35° C. Improvements in enzymatic activity can be at least 1.2 times, 1.5 times, 2 times., 3 times, 5 times, 10 times or more the amidase activity of a reference amidase enzyme, such as the polypeptide of SEQ ID NO:2 or an engineered amidase of SEQ ID NO:4 under the defined condition.

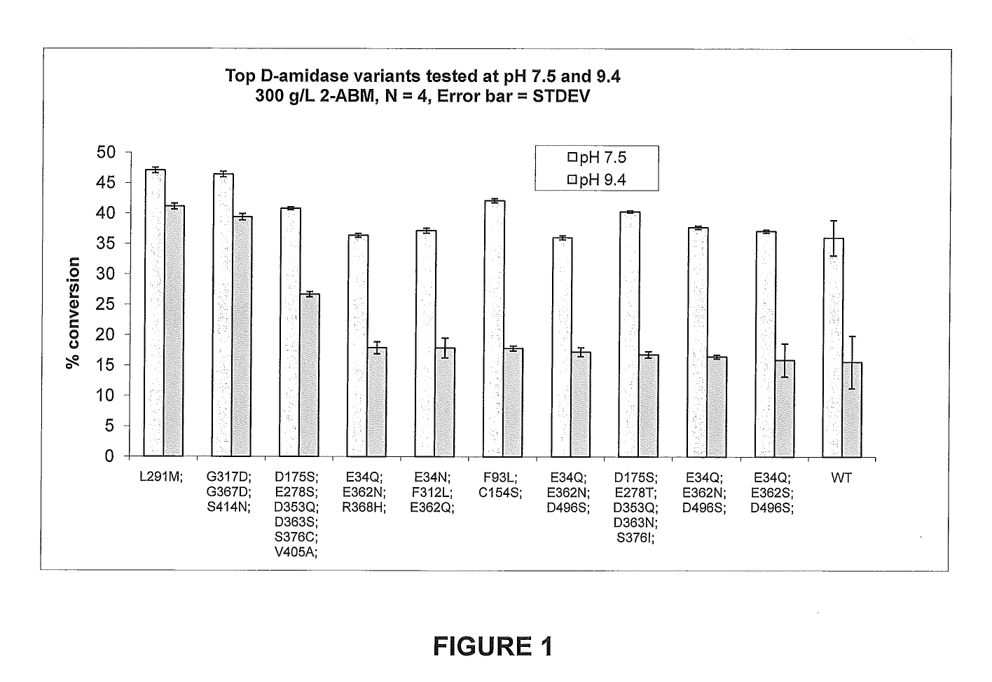

In some embodiments, the improved property of the amidase polypeptide is with respect to an increase in pH stability under defined conditions relative to a reference amidase (e.g., SEQ ID NO:2). In some embodiments, the pH stability can be reflected in enzymatic activity at an alkaline condition (e.g., about pH 9.5), where the differences in enzymatic activity can be at least 1.5 times, 2 times, 3 times, 5 times, 10 times, or more than the activity displayed by the polypeptide of SEQ ID NO:2, or an engineered amidase of SEQ ID NO:4, under the same defined alkaline pH conditions. An exemplary illustration of variant amidases having increased pH stability is shown by the results depicted in FIG. 1.

In some embodiments, the improved enzymatic activity of the amidase polypeptides can be an increase in the conversion rate of the substrate to product, such as improved conversion of a racemic mixture of 2-aminobutyramide to the corresponding (R)-2-aminobutyrate under a defined condition. In some embodiments, the defined condition comprises 475 g/L of a racemic mixture of 2-aminobutyramide under reaction conditions of pH of about 7.5 and about 35° C. in a reaction time of about 8 hrs with about 1 g/L of an amidase polypeptide. In some embodiments, the improved amidase polypeptides are capable of converting the racemic mixture of 2-aminobutyramide to the (R)-2-aminobutyrate at a yield of at least 40%, 45%, 46%, 47%, 48%, 49% or more up to the theoretical yield of 50% of the racemic mixture under the defined condition. An exemplary illustration of variant amidases having increased conversion rate is shown by the results depicted in FIG. 1.

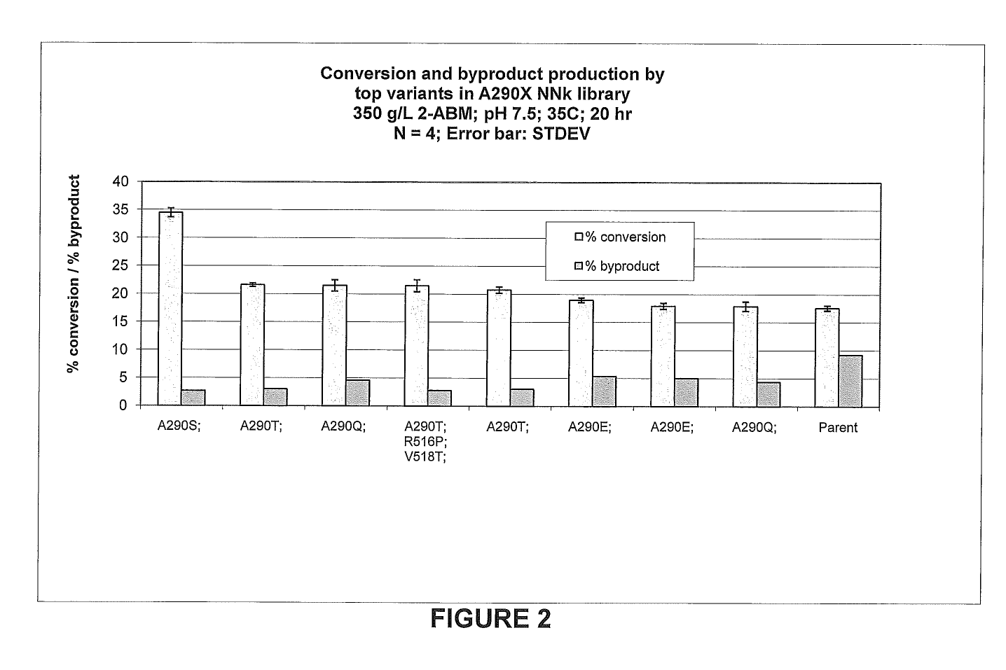

In some embodiments, the improved amidase is improved with respect to the conversion rate at a defined alkaline reaction condition. In some embodiments, the alkaline reaction condition comprises 300 g/L racemic mixture of 2-aminobutyramide under reaction conditions of pH of about 9.5 and about 25° C. in a reaction time of about 20 hrs with about 1 g/L of an amidase polypeptide. In some embodiments, the improved amidase polypeptides are capable of converting a racemic mixture of 2-aminobutyramide to the (R)-2-aminobutyrate at a yield of at least 40%, 45%, 46%, 47%, 48%, 49% or more up to the theoretical yield of 50% of the racemic mixture under the defined alkaline condition. An exemplary illustration of variant amidases having increased conversion is shown by the results depicted in FIGS. 1 and 2.

In some embodiments, the improved property of the amidase polypeptide is with respect to an increase in the stereospecificity of the enzyme for the (R)-amino acid amides, as reflected in an increase in enantiomeric excess of the corresponding (S)-amino acid amides (e.g., (S)-2-aminobutyramide over the (R)-2-aminobutyramide). In some embodiments, the increase in stereospecificity can results in an enantiomeric excess of at least 99%, 99.1%, 99.2%, 99.3%, 99.4%, 99.5%, 99.6%, 99.7%, 99.8%, or 99.9% or more of the (S)-2-aminobutyramide over the (R)-2-aminobutyramide.

In some embodiments, an improved property of the amidase is with respect to a decrease in byproducts formed in the conversion or hydrolysis of an amino acid amide substrate to the corresponding amino acid as compared to the amount of byproduct formed by a reference amidase, such as the polypeptide of SEQ ID NO:2. For the substrate (R)-2-aminobutyramide, such as in a racemic mixture of (R) and (S) forms, the amount of byproduct is less than 10%, less that 7.5%, less than 6%, less than 5%, less than 4%, less than 3%, less than 2%, less than 1%, less than 0.5%, less than 0.2%, or less than 0.1% of the total products as determined by HPLC with fluorometric detection of products following derivatization with amine reacting reagent O-phthalaldehyde (OPA) (see e.g., Example 8) (“total products” includes all compounds derivatized by OPA including 2-ABM, 2-ABA, and byproducts such as the dimer). An exemplary illustration of variant amidases having decreased percent byproduct formation relative to a wild-type amidase is shown by the results depicted in FIGS. 2-4.

In some embodiments, the amount of byproduct is reduced by at least 25% as compared to the wild-type enzyme of SEQ ID NO:2 or another engineered amidase, such as SEQ ID NO:4. In some embodiments, the amount of byproduct is reduced by at least 50%, 60%, 75%, 80%, 85%, 90% or 95% or more as compared to the wild-type enzyme of SEQ ID NO:2 or another engineered amidase, such as SEQ ID NO:4.

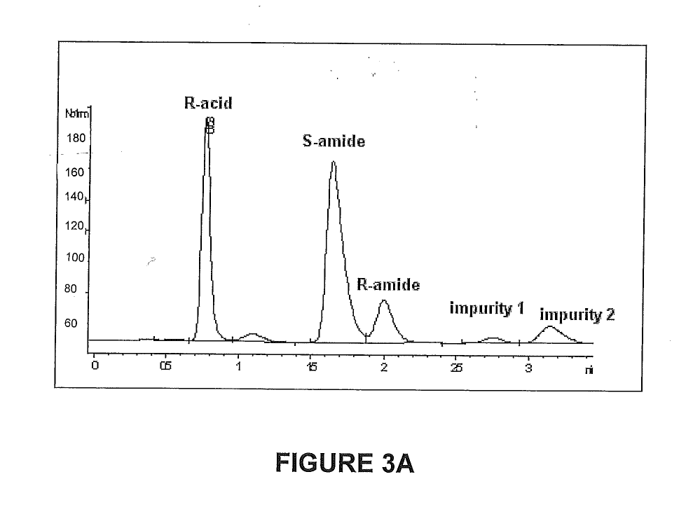

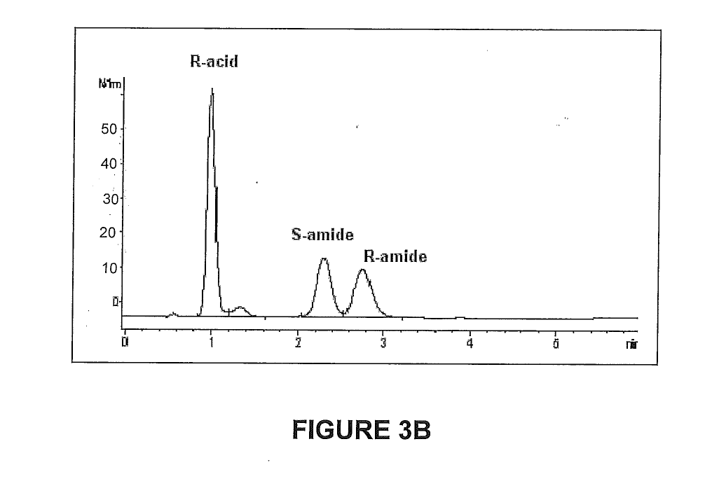

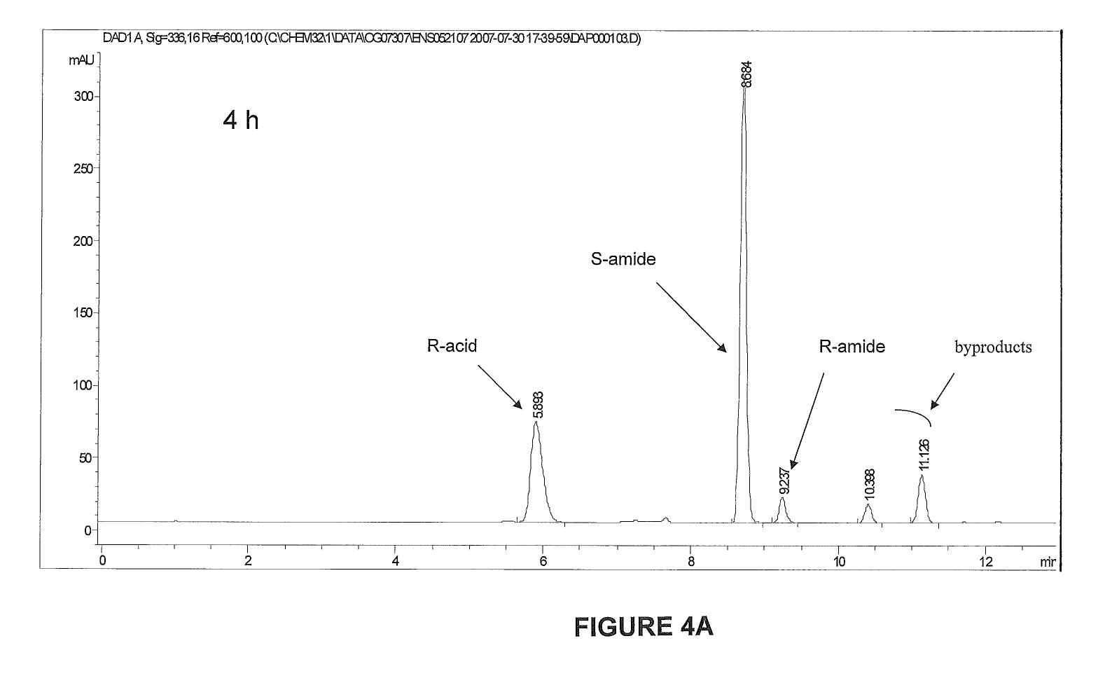

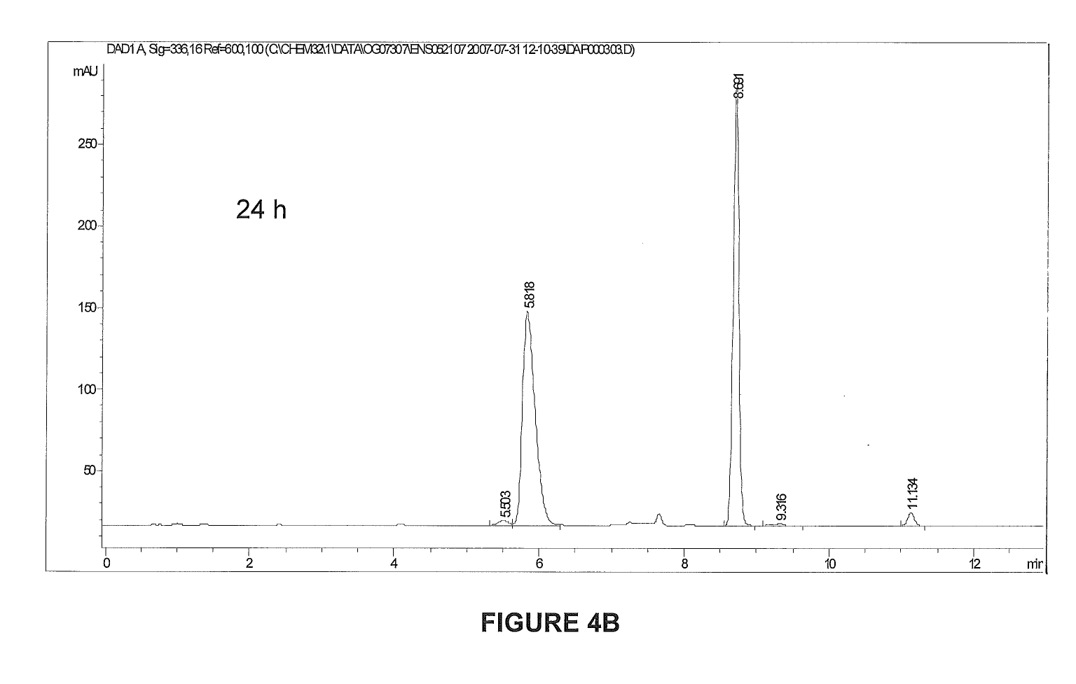

In some embodiments, the byproduct produced in the reaction mediated by amidases has a chromatographic pattern (e.g., retention profile) following derivatization with OPA and separation on RP 80A column with acetonitrile/40 mM ammonium acetate pH 5.0 (18/82) as eluent (see e.g., Examples 3 and 8, and FIGS. 3A and 4A).

In some embodiments, the byproduct as defined by mass spectroscopy (MS) has structure of formula (IX):

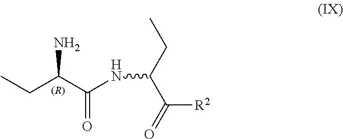

where R² can be NH₂ or OH.

In some embodiments, the improved amidase polypeptides herein can comprise an amino acid sequence that has at least about 80%, 85%, 86%, 87%, 88%, 89%, 90%, 91%, 92%, 93%, 94%, 95%, 96%, 97%, 98%, 99% or more identity as compared to a reference sequence based on SEQ ID NO:2 having at the residue corresponding to X290 a polar or acidic residue, particularly serine, threonine, glutamine, or glutamic acid, or a reference sequence of an engineered amidase, such as SEQ ID NO: 10, 12, 14, 16, 18, 20 or 22, and which has one or more of the improved properties described above.

In some embodiments, the improved amidase polypeptides herein can comprise an amino acid sequence that has at least about 80%, 85%, 86%, 87%, 88%, 89%, 90%, 91%, 92%, 93%, 94%, 95%, 96%, 97%, 98%, 99% or more identity as compared to a reference sequence based on SEQ ID NO:2 having at the residue corresponding to X290 a polar or acidic residue, particularly serine, threonine, glutamine, or glutamic acid, or a reference sequence of an engineered amidase, such as SEQ ID NO: 10, 12, 14, 16, 18, 20 or 22, and which has at least 1.5 times the activity of the polypeptide of SEQ ID NO:2 at a reaction condition of pH of about 9.5 and temperature of about 25° C. In some embodiments, the improved amidase polypeptides have at least 1.2 times the activity of SEQ ID NO: 2 at reaction condition of pH of about 7.5 and temperature of about 35° C.

In some embodiments, the improved amidase polypeptides can have residue differences in one or more residue positions as compared to the sequence of SEQ ID NO:2 at residue positions corresponding to the following: X38; X149; X175; X278; X290; X291; X315; X317; X353; X363; X367; X376; X405; X414; X516; and X518. In some embodiments, the amidase polypeptides can have additionally 1-2, 1-3, 1-4, 1-5, 1-6, 1-7, 1-8,1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other amino acid residue positions not defined by X above. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other amino acid residue positions. In some embodiments, the residue differences at other residue positions comprise conservative mutations.

In some embodiments, the amino acid residues at the specified residue positions can be selected from the following features: residue corresponding to X38 is a basic residue; residue corresponding to X149 is a polar residue; residue corresponding to X175 is a polar residue; residue corresponding to X278 is a polar residue; residue corresponding to X290 is a polar or acidic residue; residue corresponding to X291 is a non-polar or aliphatic residue; residue corresponding to X315 is a non-polar residue; residue corresponding to X317 is an acidic residue; residue corresponding to X353 is polar residue; residue corresponding to X363 is a polar residue; residue corresponding to X367 is an acidic residue; residue corresponding to X376 is cysteine (C) or an aliphatic residue; residue corresponding to X405 is an aliphatic residue; residue corresponding to X414 is a polar residue; residue corresponding to X516 is a constrained residue; and residue corresponding to X518 is a polar residue.

In some embodiments, the amino acid residues at the specified residue positions can be selected from the following features: residue corresponding to X38 is arginine or lysine, particularly arginine; residue corresponding to X149 is threonine, asparagine or glutamine, particularly threonine; residue corresponding to X175 is serine, threonine, asparagine, or glutamine, particularly serine; residue corresponding to X278 is serine, threonine, asparagine or glutamine, particularly serine or threonine; residue corresponding to X290 is serine, threonine, asparagine, glutamine, aspartic acid, or glutamic acid, particularly serine, threonine, glutamine, or glutamic acid; residue corresponding to X291 is glycine, methionine, alanine, valine, or isoleucine, particularly methionine; residue corresponding to X315 is glycine, methionine, alanine, valine, leucine or isoleucine, particularly glycine; residue corresponding to X317 is glutamic acid or aspartic acid, particularly aspartic acid; residue corresponding to X353 is serine, threonine, asparagine or glutamine, particularly glutamine; residue corresponding to X363 is serine, threonine, asparagine or glutamine, particularly serine; residue corresponding to X367 is glutamic acid or aspartic acid, particularly aspartic acid; residue corresponding to X376 is cysteine, alanine, valine, leucine, or isoleucine, particularly isoleucine or cysteine; residue corresponding to X405 is alanine, leucine, or isoleucine, particularly alanine; residue corresponding to X414 is threonine, asparagine or glutamine, particularly asparagine; residue corresponding to X516 is proline or histidine, particularly proline; and residue corresponding to X518 is serine, threonine, asparagine, or glutamine, particularly threonine.

In some embodiments, the improved amidase polypeptide comprises an amino acid sequence that includes at least one or more of the following features: residue corresponding to X290 is a polar or acidic residue; residue corresponding to X291 is non-polar or aliphatic residue; residue corresponding to X317 is an acidic residue; residue corresponding to X367 is an acidic residue; and residue corresponding to X414 is a polar residue.

In some embodiments, the improved amidase polypeptide comprises an amino acid sequence that includes at least the following features: residue corresponding to X290 a polar or acidic residue. In some embodiments, the improved amidase polypeptide comprises an amino acid sequence that includes at least the following feature: residue corresponding to X290 is a serine, threonine, glutamine, asparagine, glutamic acid or aspartic acid, and particularly serine, threonine, glutamine or glutamic acid. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations. In some embodiments, the engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% identical to a reference amino acid sequence based on SEQ ID NO:2 having the features described herein for residue corresponding to X290 with the proviso that the engineered amidase polypeptides have at the residue corresponding to X290 at least the preceding features.

In some embodiments, the improved amidase polypeptide comprises an amino acid sequence that includes at least the following features: residue corresponding to X291 is a non-polar or aliphatic residue. In some embodiments, the residue corresponding to X291 is glycine, methionine, alanine, valine, or isoleucine, particularly a methionine. In some embodiments, the amidase polypeptides can have additionally 1-2, 1-3, 1-4, 1-5, 1-6, 1-7, 1-8,1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations. In some embodiments, the engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% identical to a reference amino acid sequence based on SEQ ID NO:2 having the features described herein for the residue corresponding to X291 with the proviso that the engineered amidase polypeptides have at the residue corresponding to X291 at least the preceding features (e.g., SEQ ID NO:6).

In some embodiments, the improved amidase polypeptide comprises an amino acid sequence that includes at least the following features: residue corresponding to X317 is an acidic residue; residue corresponding to X367 is an acidic residue; and residue corresponding to X414 is a polar residue. In some embodiments, the improved amidase polypeptide comprises an amino acid sequence that includes at least the following features: residue corresponding to X317 is aspartic or glutamic acid, particularly aspartic acid; residue corresponding to X367 is aspartic or glutamic acid, particularly aspartic acid; and residue corresponding to X414 is threonine, asparagine or glutamine, particularly asparagine. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions not defined by X above. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 50, 55, or 60 residue differences at the other residue positions. In some embodiments, the residue differences at other residue positions comprise conservative mutations. In some embodiments, the engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% identical to a reference amino acid sequence based on SEQ ID NO:2 having the features described herein for the residues corresponding to X317, X367 and X414, with the proviso that the engineered amidase polypeptides have at the residues corresponding to X317, X367 and X414 at least the preceding features (e.g., SEQ ID NO:8).

In some embodiments, the improved amidase polypeptide comprises an amino acid sequence that includes at least the following features: residue corresponding to X290 is a polar or acidic residue; residue corresponding to X317 is an acidic residue; residue corresponding to X367 is an acidic residue; and residue corresponding to X414 is a polar residue. In some embodiments, the amidase polypeptides comprises an amino acid sequence that includes at least the following features: residue corresponding to X290 is serine, threonine, asparagine, glutamine, glutamic acid, or aspartic acid, particularly serine, threonine, glutamine or glutamic acid; residue corresponding to X317 is aspartic or glutamic acid, particularly aspartic acid; residue corresponding to X367 is aspartic or glutamic acid, particularly aspartic acid; and residue corresponding to X414 is threonine, asparagine, or glutamine, particularly asparagine. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations. In some embodiments, the engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% identical to a reference amino acid sequence based on SEQ ID NO:2 having the features described herein for the residues corresponding to X290, X317, X367 and X414, with the proviso that the engineered amidase polypeptides have at the residues corresponding to X290, X317, X367, and X414 at least the preceding features (e.g., SEQ ID NO: 10, 12, 14, or 16).

In some embodiments, the improved amidase polypeptide comprises an amino acid sequence that includes at least the following features: residue corresponding to X290 is threonine; residue corresponding to X317 is aspartic acid; residue corresponding to X367 is aspartic acid; and residue corresponding to X414 is asparagine. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions as compared to SEQ ID NO:2. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations. In some embodiments, the engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% identical to a reference amino acid sequence based on SEQ ID NO:2 having the features described herein for the residues corresponding to X290, X317, X367, and X414, with the proviso that the engineered amidase polypeptides have at the residues corresponding to X290, X317, X367, and X414 at least the preceding features (e.g., SEQ ID NO:10).

In some embodiments, the amidase polypeptide comprises an amino acid sequence that includes at least the following features: residue corresponding to X290 is glutamine; residue corresponding to X317 is aspartic acid; residue corresponding to X367 is aspartic acid; and residue corresponding to X414 is asparagine. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions as compared to SEQ ID NO:2. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations. In some embodiments, the engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% identical to a reference amino acid sequence based on SEQ ID NO:2 having the features described herein for the residues corresponding to X290, X317, X367 and X414, with the proviso that the engineered amidase polypeptides have at the residues corresponding to X290, X317, X367, and X414 at least the preceding features (e.g., SEQ ID NO:12).

In some embodiments, the amidase polypeptide comprises an amino acid sequence that includes at least the following features: residue corresponding to X290 is glutamic acid; residue corresponding to X317 is aspartic acid; residue corresponding to X367 is aspartic acid; and residue corresponding to X414 is asparagine. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions as compared to SEQ ID NO:2. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations. In some embodiments, an engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% identical to a reference amino acid sequence based on SEQ ID NO:2 having the features described herein for the residues corresponding to X290, X317, X367, and X414, with the proviso that the engineered amidase polypeptides have at the residues corresponding to X290, X317, X367, and X414 at least the preceding features (e.g., SEQ ID NO:14).

In some embodiments, the amidase polypeptide comprises an amino acid sequence that includes at least the following features: residue corresponding to X290 is serine; residue corresponding to X317 is aspartic acid; residue corresponding to X367 is aspartic acid; and residue corresponding to X414 is asparagine. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions as compared to SEQ ID NO:2. In some embodiments, the residue differences at other residue positions comprise conservative mutations. In some embodiments, an engineered amidase polypeptide can comprise an amino acid sequence that is at least about 80%, 85%, 86%, 87%, 88%, 89%, 90%, 91%, 92%, 93%, 94%, 95%, 96%, 97%, 98%, 99% identical to a reference amino acid sequence based on SEQ ID NO:2 having the features described herein for the residues corresponding to X290, X317, X367, and X414, with the proviso that the engineered amidase polypeptides have at the residues corresponding to X290, X317, X367, and X414 at least the preceding features (e.g., SEQ ID NO:16).

In some embodiments, the improved amidase polypeptides comprising an amino acid sequence having the specified features or combination of features for residues X290, X291, X317, X367, and/or X414 as provided in the foregoing, can further include one or more residue differences as compared to SEQ ID NO:2 at the following residue positions: residue corresponding to X38: residue corresponding to X149; residue corresponding to X175; residue corresponding to X278; residue corresponding to X315; residue corresponding to X353; residue corresponding to X363; residue corresponding to X376; residue corresponding to X405; residue corresponding to X516; and residue corresponding to X518.

In some embodiments, the amino acid residues at the specified residue positions in the forgoing can be selected from the following features: residue corresponding to X38 is a basic residue; residue corresponding to X149 is a polar residue; residue corresponding to X175 is a polar residue; residue corresponding to X278 is a polar residue; residue corresponding to X315 is a non-polar residue; residue corresponding to X353 is a polar residue; residue corresponding to X363 is a polar residue; residue corresponding to X376 is cysteine or an aliphatic residue; residue corresponding to X405 is an aliphatic residue; residue corresponding to X516 is a constrained residue; and residue corresponding to X518 is a polar residue. The specific amino acids for each of the specified residue positions can be selected from the amino acid residues described previously for these residue positions.

In some embodiments, the improved amidase polypeptides comprising an amino acid sequence having the specified features or combination of features for residues X290, X291, X317, X367, and/or X414 as describe above, can have additionally at the residue corresponding to X38 a basic residue. In some embodiments, residue corresponding to X38 is arginine or lysine, particularly arginine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have additionally at the residue corresponding to X149 a polar residue. In some embodiments, the residue corresponding to X149 is an asparagine, glutamine, or threonine, particularly threonine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have additionally at the residue corresponding to X175 a polar residue. In some embodiments, the residue corresponding to X175 is an asparagine, glutamine, threonine, or serine, particularly serine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8,1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have additionally at the residue corresponding to X278 a polar residue. In some embodiments, the residue corresponding to X278 is an serine, threonine, asparagine, or glutamine, particularly serine or threonine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8,1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have additionally at the residue corresponding to X315 a non-polar residue. In some embodiments, the residue corresponding to X315 is glycine, methionine, alanine, valine, leucine or isoleucine, particularly glycine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have additionally at the residue corresponding to X353 a polar residue. In some embodiments, the residue corresponding to X353 is serine, threonine, asparagine, or glutamine, particularly glutamine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have additionally at the residue corresponding to X363 a polar residue. In some embodiments, residue corresponding to X363 is an serine, threonine, asparagine, or glutamine, particularly asparagine or serine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have additionally at the residue corresponding to X376 a cysteine or an aliphatic residue. In some embodiments, the residue corresponding to X376 is cysteine, alanine, valine, leucine or isoleucine, particularly cysteine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have at the residue corresponding to X405 an aliphatic residue. In some embodiments, the residue corresponding to X405 is alanine, leucine or isoleucine, particularly alanine. In some embodiments, the amidase polypeptides comprising an amino acid sequence having the preceding features can have additionally one or more residue differences at other amino acid residues as compared to a reference sequence of SEQ ID NO:2. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have at the residue corresponding to X516 a constrained residue. In some embodiments, the residue corresponding to X516 is a histidine or proline, particularly proline. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides comprising an amino acid sequence having the specified features or combination of features for residues X290, X291, X317, X367, and/or X414 as describe above, can have at the residue corresponding to X518 a polar residue. In some embodiments, the residue corresponding to X518 is an asparagine, glutamine, serine or threonine, particularly serine or threonine. In some embodiments, the amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions. In some embodiments, the residue differences at other residue positions comprise conservative mutations.

In some embodiments, the improved amidase polypeptides having the specified features or combination of features for residues X290, X291, X317, X367, and/or X414 as describe above, comprises an amino acid sequence that further includes one or more of the following features: residue corresponding to X38 is basic residue; residue corresponding to X149 is a polar residue; residue corresponding to X516 is a constrained residue; and residue corresponding to X518 is a polar residue. In some embodiments, the improved amidase polypeptides can comprises an amino acid sequence, that in addition to the preceding features, further comprises one or more of the following features: residue corresponding to X175 is a polar residue; residue corresponding to X278 is a polar residue; residue corresponding to X315 is non-polar residue corresponding to X353 is polar residue; residue corresponding to X363 is a polar residue; residue corresponding to X376 is cysteine (C) or an aliphatic residue; and residue corresponding to X405 is an aliphatic residue.

In some embodiments, the improved amidase polypeptides having the specified features or combination of features for residues X290, X291, X317, X367, and/or X414 as describe above, comprises an amino acid sequence that further includes one or more of the following features: residue corresponding to X38 is arginine or lysine, particularly arginine; residue corresponding to X149 is threonine, asparagine, or glutamine, particularly threonine; residue corresponding to X516 is a proline or histidine, particularly proline; and residue corresponding to X518 is serine, threonine, asparagine, or glutamine, particularly threonine. In some embodiments, the improved amidase polypeptides can comprises an amino acid sequence, that in addition to the preceding features, further comprises one or more of the following features: residue corresponding to X175 is serine, threonine, asparagine, or glutamine, particularly serine; residue corresponding to X278 is serine, threonine, asparagine, or glutamine, particularly serine; residue corresponding to X315 is glycine, methionine, alanine, valine, leucine, or isoleucine, particularly glycine; residue corresponding to X353 is serine, threonine, asparagine, or glutamine, particularly glutamine; residue corresponding to X363 is serine, threonine, asparagine, or glutamine, particularly serine; residue corresponding to X376 is cysteine or alanine, valine, leucine, or isoleucine, particularly cysteine; and residue corresponding to X405 is alanine, leucine, or isoleucine, particularly alanine.

In some embodiments, the improved amidase polypeptide comprises an amino acid sequence that includes at least the following features: residue corresponding to X290 is a polar or acidic residue; residue corresponding to X317 is an acidic residue; residue corresponding to X367 is an acidic residue; and residue corresponding to X414 is a polar residue; residue corresponding to X516 is a constrained residue; and residue corresponding to X518 is a polar residue. In some embodiments, the improved amidase polypeptide comprises an amino acid sequence that includes at least the following features: the residue corresponding to X290 is serine, threonine, asparagine, glutamine, aspartic acid, or glutamic acid, particularly serine, threonine, glutamine, or glutamic acid; residue corresponding to X317 is aspartic or glutamic acid, particularly aspartic acid; residue corresponding to X367 is aspartic or glutamic acid, particularly aspartic acid; residue corresponding to X414 is threonine, asparagine, or glutamine, particularly asparagine; residue corresponding to X516 is proline or histidine, particularly proline; and residue corresponding to X518 is serine, threonine, asparagine or glutamine, particularly threonine. In some embodiments, these amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other amino acid residue positions as compared to the reference sequence of SEQ ID NO:18. In some embodiments, the number of residue differences can be 1, 2, 3, 4, 5, 6, 7, 8, 9, 10, 11, 12, 14, 15, 16, 18, 20, 22, 24, 26, 30, 35, 40, 45, 50, 55, or 60 differences at other residue positions. In some embodiments, the residue differences comprise conservative mutations. In some embodiments, the amidase polypeptide comprises an amino acid sequence that has at least 80%, 85%, 86%, 87%, 88%, 89%, 90%, 91%, 92%, 93%, 94%, 95%, 96%, 97%, 98%, or 99% identity to a reference sequence based on SEQ ID NO:2 having the preceding features at the specified residue positions, with the proviso that the amidase amino acid sequence has at least the preceding features (e.g., SEQ ID NO:18).

In some embodiments, the improved amidase polypeptide comprises an amino acid sequence that includes at least the following features: residue corresponding to X38 is a basic residue; residue corresponding to X149 is a polar residue; residue corresponding to X290 is a polar or acidic residue; residue corresponding to X317 is an acidic residue; residue corresponding to X367 is an acidic residue; and residue corresponding to X414 is a polar residue. In some embodiments, the improved amidase polypeptide comprises an amino acid sequence that includes at least the following features: residue corresponding to X38 is glutamine or arginine, particularly arginine; residue corresponding to X149 is threonine, asparagine or glutamine, particularly threonine; residue corresponding to X290 is a serine, threonine, asparagine, glutamine, aspartic acid, or glutamic acid, particularly serine, threonine, glutamine or glutamic acid; residue corresponding to X317 is aspartic or glutamic acid, particularly aspartic acid; residue corresponding to X367 is aspartic or glutamic acid, particularly aspartic acid; and residue corresponding to X414 is threonine, asparagine, or glutamine, particularly threonine. In some embodiments, these amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other amino acid residue positions as compared to the reference sequence of SEQ ID NO:20. In some embodiments, the number of residue differences can be 1, 2, 3, 4, 5, 6, 7, 8, 9, 10, 11, 12, 14, 15, 16, 18, 20, 22, 24, 26, 30, 35, 40, 45, 50, 55, or 60 differences at other residue positions. In some embodiments, the residue differences comprise conservative mutations. In some embodiments, the amidase polypeptide comprises an amino acid sequence that has at least 80%, 85%, 86%, 87%, 88%, 89%, 90%, 91%, 92%, 93%, 94%, 95%, 96%, 97%, 98%, or 99% identity to a reference sequence based on SEQ ID NO:2 having the preceding features at the specified residue positions, with the proviso that the amidase amino acid sequence has at least the preceding features (e.g. SEQ ID NO:20).

In some embodiments, the improved amidase polypeptide comprises an amino acid sequence that includes at least the following features: residue corresponding to X175 is a polar residue; residue corresponding to X278 is a polar residue; residue corresponding to X353 is a polar residue; residue corresponding to X363 is a polar residue; residue corresponding to X376 is cysteine; and residue corresponding to X405 is an aliphatic residue. In some embodiments, the improved amidase polypeptide comprises an amino acid sequence that includes at least the following features: residue corresponding to X175 is serine, threonine, asparagine, or glutamine, particularly serine; residue corresponding to X278 is serine, threonine, asparagine, or glutamine, particularly serine; residue corresponding to X353 is serine, threonine, asparagine, or glutamine, particularly glutamine; residue corresponding to X363 is serine, threonine, asparagine, or glutamine, particularly serine; residue corresponding to X376 is cysteine; and residue corresponding to X405 is alanine, leucine, or isoleucine, particularly alanine. In some embodiments, these amidase polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions as compared to a reference of SEQ ID NO:22. In some embodiments, the number of residue differences can be 1, 2, 3, 4, 5, 6, 7, 8, 9, 10, 11, 12, 14, 15, 16, 18, 20, 22, 24, 26, 30, 35, 40, 45, 50, 55, or 60 differences at other residue positions. In some embodiments, the residue differences comprise conservative mutations. In some embodiments, the amidase polypeptide comprises an amino acid sequence that has at least 80%, 85%, 86%, 87%, 88%, 89%, 90%, 91%, 92%, 93%, 94%, 95%, 96%, 97%, 98%, or 99% identity to a reference sequence based on SEQ ID NO:2 having the preceding features at the specified residue positions, with the proviso that the amidase amino acid sequence has at least the preceding features (e.g., SEQ ID NO:22).

Table 2 below lists engineered amidase polypeptides (and encoding polynucleotides) by sequence identifier (SEQ ID NO) disclosed herein together with the specific residue differences of the variant sequences of the engineered polypeptides with respect to the wild-type Ochrobactrum anthropi amidase polypeptide sequence (SEQ ID NO:2) from which they were derived by directed evolution (see e.g., Stemmer et al., 1994, Proc Natl Acad Sci USA 91:10747-10751). Each row of Table 2 lists two SEQ ID NOs, where the odd number refers to the nucleotide sequence that encodes for the polypeptide amino acid sequence provided by the even number.

The activity of each engineered amidase polypeptide was determined relative to either the wild-type (SEQ ID NO: 2) or the E315G mutant (SEQ ID NO: 4), which both exhibit about the same activity. Activity was determined as conversion of a racemic mixture of 2-aminobutyramide to (R)-2-aminobutyrate and (S)-2-aminobutyramide at pH 9.5 at 25° C., as described in Example 5. The activity was quantified as follows: “+” indicates that the engineered amidase activity is from 1.5 times to 3 times the activity of the polypeptide of SEQ ID NO:2 or SEQ ID NO:4; and “++” indicates that the engineered amidase activity is from 3 times to about 5 times the activity of SEQ ID NO:2 or SEQ ID NO:4.

TABLE 2

| nt/aa | | |
|---|---|---|
| SEQ ID NO: | Residue Difference(s) Relative to SEQ ID NO: 2 | Activity |
| 3/4 | E315G | − |
| 5/6 | L291M; E315G | + |
| 7/8 | E315G; G317D; G367D; S414N | + |
| 9/10 | A290T; E315G; G317D; G367D; S414N | + |
| 11/12 | A290Q; E315G; G317D; G367D; S414N | + |
| 13/14 | A290E; E315G; G317D; G367D; S414N | + |
| 15/16 | A290S; E315G; G317D; G367D; S414N | ++ |
| 17/18 | A290T; E315G; G317D; G367D; S414N; | ++ |
| | R516P; V518T | |
| 19/20 | Q38R; S149T; A290S; E315G; G317D; | ++ |
| | G367D; S414N; | |
| 21/22 | D175S; E278S; E315G; D353Q; D363S; | ++ |
| | S376C; V405A | |

In some embodiments, the improved amidase polypeptide can comprise an amino acid sequence that is at least 80%, 85%, 86%, 87%, 88%, 89%, 90%, 91%, 92%, 93%, 94%, 95%, 96%, 97%, 98%, or 99% identical to a reference amino acid sequence of any one of SEQ ID NO:8, 10, 12, 14, 16, 18, 20, and 22. In some embodiments, these amidase polypeptides can have additionally from about 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences as compared to the reference sequence. In some embodiments, the number of residue differences can be 1, 2, 3, 4, 5, 6, 7, 8, 9, 10, 11, 12, 14, 15, 16, 18, 20, 22, 24, 26, 30, 35, 40, 45, 50, 55, or 60 differences as compared to the reference sequence. The residue differences can comprise insertions, deletions, or substitutions, or combinations thereof. In some embodiments, the residue differences comprise conservative substitutions as compared to the reference sequence.

In some embodiments, the improved amidase polypeptide can comprise an amino acid sequence that is at least 80%, 85%, 86%, 87%, 88%, 89%, 90%, 91%, 92%, 93%, 94%, 95%, 96%, 97%, 98%, or 99% identical to a reference amino acid sequence of any one of SEQ ID NO:8, 10, 12, 14, 16, 18, 20, and 22, with the proviso that the amidase amino acid sequence includes any one set of the substitution mutations provided in Table 2. In some embodiments, these amidase polypeptides can have additionally from about 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions as compared to the reference sequence. In some embodiments, the number of residue differences can be 1, 2, 3, 4, 5, 6, 7, 8, 9, 10, 11, 12, 14, 15, 16, 18, 20, 22, 24, 26, 30, 35, 40, 45, 50, 55, or 60 differences as compared to the reference sequence. The residue differences can comprise insertions, deletions, or substitutions, or combinations thereof. In some embodiments, the residue differences comprise conservative substitutions as compared to the references sequence.

In some embodiments, an improved amidase comprises an amino acid sequence corresponding to SEQ ID NO: 6, 8, 10, 12, 14, 16, 18, 20, or 22.

In some embodiments, the improved amidase polypeptide is capable of stereospecifically converting (R)-2-aminobutyramide in a racemic mixture of 2-aminobutyramide to (R)-2-aminobutyrate, with (S)-2-aminobutyramide in at least 90%, 91%, 92%, 93%, 94%, 95%, 96%, 97%, 98%, or 99% or greater stereomeric excess. In some embodiments, the amidase polypeptide capable of converting (R)-2-aminobutyramide in a racemic mixture of 2-aminobutyramide to (R)-2-aminobutyrate, with (S)-2-aminobutyramide in at least 90%, 91%, 92%, 93%, 94%, 95%, 96%, 97%, 98%, or 99% or greater stereomeric excess comprises an amino acid sequence selected from SEQ ID NO: 6, 8, 10, 12, 14, 16, 18, 20, and 22.

In some embodiments, the improved amidase polypeptide is capable of stereospecifically converting at least 40%, 45%, 46%, 47%, 48% or 49% of about 475 g/L of a racemic mixture of 2-aminobutyramide to (R)-2-aminobutyrate under reaction conditions of pH of about 7.5 and about 35° C. in about 8 hrs with about 1 g/L of amidase polypeptide. In some embodiments, the improved amidase polypeptide capable of stereospecifically converting at least 40%, 45%, 46%, 47%, 48% or 49% of 475 g/L of a racemic mixture of 2-aminobutyramide to (R)-2-aminobutyrate under reaction conditions of pH of about 7.5 and about 35° C. in about 8 hrs comprises an amino acid sequence selected from SEQ ID NO: 6, 8, 10, 12, 14, 16, 18, 20, and 22.

In some embodiments, the improved amidase polypeptide is capable of stereospecifically converting at least 40%, 45%, 46%, 47%, 48% or 49% of about 300 g/L of a racemic mixture of 2-aminobutyramide to (R) 2-aminobutyrate under reaction conditions of pH of about 9.5 and about 25° C. in about 20 hrs. In some embodiments, the improved amidase polypeptide capable of converting at least 40%, 45%, 46%, 47%, 48% or 49% of about 300 g/L of a racemic mixture of 2-aminobutyramide to (R)-2-aminobutyrate under reaction conditions of pH of about 9.5 and about 25° C. in about 20 hrs comprises an amino acid sequence selected from SEQ ID NO: 6, 8, 10, 12, 14, 16, 18, 20, and 22.

As noted above, in some embodiments, the improved amidase polypeptide is capable of stereospecifically converting (R)-2-aminobutyramide to (R)-2-aminobutyrate in a racemic mixture of 2-aminobutyramide with reduced level of byproduct as compared to the activity of SEQ ID NO: 2. In some embodiments, the amidase polypeptide is capable of carrying out the conversion reaction such that the percentage of byproducts is less than 10%, less than 7.5%, less than 6%, less than 5%, less than 4%, less than 3%, less than 2%, less than 1%, less than 0.5%, less than 0.2%, or less than 0.1% of the total products, as defined above. In some embodiments, the improved amidase polypeptide capable of converting (R)-2-aminobutyramide to (R)-2-aminobutyrate in a racemic mixture of 2-aminobutyramide with reduced level of byproduct as compared to the activity of SEQ ID NO:2 comprises an amino acid sequence selected from SEQ ID NO: 8, 10, 12, 14, 16, 18, 20, and 22.

The present invention further provides an engineered amidase polypeptide having an amino acid sequence selected from the group consisting of: (a) an amino acid sequence that is at least 80% identical to SEQ ID NO: 2 wherein the amino acid sequence comprises one or more features selected from the group consisting of: residue corresponding to X38 is arginine; residue corresponding to X149 is threonine; residue corresponding to X175 is serine, residue corresponding to X278 is threonine or serine; residue corresponding to X290 is serine, threonine, glutamine, or glutamic acid; residue corresponding to X291 is methionine, residue corresponding to X315 is glycine; residue corresponding to X353 is glutamine; residue corresponding to X363 is serine; residue corresponding to X367 is aspartic acid; residue corresponding to X376 is isoleucine or cysteine; residue corresponding to X405 is asparagine; residue corresponding to X516 is proline; and residue corresponding to X518 is threonine, where position (X) refers to the corresponding position in SEQ ID NO 2; and (b) an amino acid sequence that is encoded by a nucleic acid that hybridizes under high stringency conditions to the complement of SEQ ID NO: 1 (the polynucleotide sequence encoding SEQ ID NO: 2), wherein the encoded amino acid sequence comprises one or more features selected from the group consisting of: residue corresponding to X38 is arginine; residue corresponding to X149 is threonine; residue corresponding to X175 is serine, residue corresponding to X278 is threonine or serine; residue corresponding to X290 is serine, threonine, glutamine, or glutamic acid; residue corresponding to X291 is methionine, residue corresponding to X315 is glycine; residue corresponding to X353 is glutamine; residue corresponding to X363 is serine; residue corresponding to X367 is aspartic acid; residue corresponding to X376 is isoleucine or cysteine; residue corresponding to X405 is asparagine; residue corresponding to X516 is proline; and residue corresponding to X518 is threonine, where position (X) refers to the corresponding position in SEQ ID NO 2. In some embodiments, the amino acid sequences referred to in part (a) have at least 85%, 86%, 87%, 88%, 89%, 90%, 91%, 92%, 93%, 94%, 95%, 96%, 97%, 98%, or 99% identity to SEQ ID NO: 2. In some embodiments, the engineered amidase polypeptide comprises an amino acid sequence having at least two, three, four, five, or six or more of the above-described features. Typically, the amino acid sequences in the embodiments described in this paragraph comprise at least one feature selected from the group consisting of: residue corresponding to X290 is serine, threonine, glutamine, or glutamic acid; residue corresponding to X291 is methionine; residue corresponding to X315 is glycine; residue corresponding to X317 is aspartic acid; residue corresponding to X367 is aspartic acid; and residue corresponding to X414 is asparagine. In some embodiments, the amino acid sequence comprises the combination of features in which the residue corresponding to X290 is serine, threonine, glutamine, or glutamic acid and the residue corresponding to X315 is glycine.

Improved amidase polypeptides described herein may have amino acid sequences that in addition to having the features described hereinabove, further comprise one or more of the following features: residue corresponding to X58 is phenylalanine; residue corresponding to X61 is serine or glycine; residue corresponding to X62 is glycine or cysteine; residue corresponding to X65 is asparagine; residue corresponding to X69 is serine; residue corresponding to X156 is serine; residue corresponding to X278 is methionine; residue corresponding to X303 is valine; residue corresponding to X280 is aspartic acid or serine; residue corresponding to X156 is serine; residue corresponding to X280 is aspartic acid; residue corresponding to X156 is serine; residue corresponding to X280 is serine; and replacement of residues X476-X486 with glycine.

The improved amidases described herein exclude the following specific variants of SEQ ID NO: 2 which have the amino acid sequence of SEQ ID NO: 2 but which differ from SEQ ID NO: 2 only with respect to the following single, double, or indicated mutations (positions based on the wild-type amino acid sequence of SEQ ID NO: 2): (a) M58F; (b) C61S or C61G; (c) S62G or S62C; (d) K65N; (e) C69S; (f) G156S; (g) K278M; (h) E303V; (i) G280D or G280S; (j) G156S and G280D; (k) G156S and G280S; (I) K278M and E303V; (m) N275R; and/or (n) replacement of residues 476-486 with G. Further excluded from invention polynucleotide embodiments described herein are the specific polynucleotides encoding the variants of SEQ ID NO: 2 which have the amino acid sequence of SEQ ID NO: 2 but which differ from SEQ ID NO: 2 only with respect to the following single, double, or indicated mutations (where amino acid position is based on the wild-type amino acid sequence of SEQ ID NO: 2): (a) M58F; (b) C61S or C61G; (c) S62G or S62C; (d) K65N; (e) C69S; (f) G156S; (g) K278M; (h) E303V; (i) G280D or G280S; (j) G156S and G280D; (k) G156S and G280S; (I) K278M and E303V; (m) N275R; and/or (n) replacement of residues 476-486 with G.

In some embodiments, the improved engineered amidase enzymes comprise deletions of the engineered amidase polypeptides described herein. Thus, for each and every embodiment of the amidase polypeptides of the disclosure, the deletions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the amidase polypeptides, as long as the functional activity of the amidase activity is maintained. In some embodiments, the deletions can comprise, 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 amino acid residues. In some embodiments, the number of deletions can be 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 amino acids. In some embodiments, the deletions can comprise deletions of 1, 2, 3, 4, 5, 6, 7, 8, 9, 10, 11, 12, 13, 14, 15, 16, 18, 20, 22, 24, 26, 29 or 30 amino acid residues.

As described herein, the improved amidase polypeptides of the disclosure can be in the form of fusion polypeptides in which the amidase polypeptides are fused to other polypeptides, such as, by way of example and not limitation, antibody tags (e.g., myc epitope), purifications sequences (e.g., His tags), and cell localization signals (e.g., secretion signals). Thus, the amidase polypeptides can be used with or without fusions to other polypeptides.

In some embodiments, the polypeptides described herein are not restricted to the genetically encoded amino acids and may be comprised, either in whole or in part, of naturally-occurring and/or synthetic non-encoded amino acids. Certain commonly encountered non-encoded amino acids of which the polypeptides described herein may be comprised include, but are not limited to: the D-stereoisomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (Melle); phenylglycine (Phg); cyclohexylalanine (Cha); norleucine (Nle); naphthylalanine (NaI); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aOly); propargylglycine (pgGly); homoalanine (hAla); norvaline (nVal); homoleucine (hLeu), homovaline (hVal); homoisolencine (hlle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised will be apparent to those of skill in the art (see, e.g., the various amino acids provided in Fasman, 1989, CRC Practical Handbook of Biochemistry and Molecular Biology, CRC Press, Boca Raton, Fla., at pp. 3-70 and the references cited therein, all of which are incorporated by reference). These amino acids may be in either the L- or D-configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys(nitropyridinesulfenyl), Glu(δ-benzylester), Gln(xanthyl), Asn(N-δ-xanthyl), His(bom), His(benzyl), His(tos), Lys(fmoc), Lys(tos), Ser(O-benzyl), Thr (O-benzyl) and Tyr(O-benzyl).

Non-encoding amino acids that are conformationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and 1-aminocyclopentane-3-carboxylic acid.

### Polynucleotides Encoding Engineered Amidases

In another aspect, the present disclosure provides polynucleotides encoding the invention engineered amidase enzymes described herein. The polynucleotides may be operatively linked to one or more heterologous control sequences that regulate gene expression to create a recombinant polynucleotide capable of expressing the polypeptide. Expression constructs containing a heterologous polynucleotide encoding the engineered amidase can be introduced into appropriate host cells to express the corresponding amidase polypeptide.

Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved amidase enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein, including the amino acid sequences presented in Table 2. In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells.

In some embodiments, the polynucleotide comprises a nucleotide sequence encoding an amidase polypeptide comprising an amino acid sequence that has at least about 80%, 85%, 86%, 87%, 88%, 89%, 90%, 91%, 92%, 93%, 94%, 95%, 96%, 97%, 98%, or 99% or more sequence identity to any of the reference engineered amidase polypeptides described herein, e.g., SEQ ID NO:18.

For example, in some embodiments, the polynucleotide comprises a nucleotide sequence encoding an amidase polypeptide with at least about 80%, 85%, 86%, 87%, 88%, 89%, 90%, 91%, 92%, 93%, 94%, 95%, 96%, 97%, 98%, or 99% or more sequence identity to a reference amino acid sequence based on SEQ ID NO:2 having at residue corresponding to X290 a polar or acidic residue, where the encoded amidase polypeptide has at least the specified features at the residue corresponding to X290. In some embodiments, the residue corresponding to X290 is a serine, threonine, asparagine, glutamine, glutamic acid, or aspartic acid, particularly serine, threonine, glutamine, or glutamic acid.

In some embodiments, the polynucleotide comprises a nucleotide acid sequence encoding an amidase polypeptide with at least about 80%, 85%, 86%, 87%, 88%, 89%, 90%, 91%, 92%, 93%, 94%, 95%, 96%, 97%, 98%, or 99% or more sequence identity to a reference amino acid sequence based on SEQ ID NO:2 having at least at residue corresponding to X291 a non-polar or aliphatic amino acid, where the encoded amidase polypeptide has at least the specified features at the residue corresponding to X291. In some embodiments, the residue corresponding to X291 is a methionine.

In some embodiments, the polynucleotide comprises a nucleotide acid sequence encoding an amidase polypeptide with at least about 80%, 85%, 86%, 87%, 88%, 89%, 90%, 91%, 92%, 93%, 94%, 95%, 96%, 97%, 98%, or 99% or more sequence identity to a reference amino acid sequence based on SEQ ID NO:2 having at least one or more or at least all of the following features: residue corresponding to X317 is an acidic residue; residue corresponding to X367 is an acidic residue; and residue corresponding to X414 is a polar residue, where the encoded amidase polypeptide has at least the preceding features. In some embodiments, the polynucleotide encodes an improved amidase polypeptide comprising an amino acid sequence that includes one or more or at least all of the following features: residue corresponding to X317 is aspartic or glutamic acid, particularly aspartic acid; residue corresponding to X367 is aspartic or glutamic acid, particularly aspartic acid; and residue corresponding to X414 is threonine, asparagine, or glutamine, particularly asparagine.

In some embodiments, the polynucleotide comprises a nucleotide sequence encoding an amidase polypeptide with at least about 80%, 85%, 86%, 87%, 88%, 89%, 90%, 91%, 92%, 93%, 94%, 95%, 96%, 97%, 98%, or 99% or more sequence identity to a reference amino acid sequence based on SEQ ID NO:2 having at position corresponding to X290 a polar or acidic residue, and one or more or at least all of the following features: residue corresponding to X317 is an acidic residue; residue corresponding to X367 is an acidic residue; and residue corresponding to X414 is a polar residue, wherein the encoded amidase polypeptide has at least the specified amino acid residue at X290 and the preceding features. In some embodiments, the residue corresponding to X290 is serine, threonine, asparagine, glutamine, glutamic acid or aspartic acid, particularly serine, threonine, glutamine, or glutamic acid; residue corresponding to X317 is aspartic or glutamic acid, particularly aspartic acid; residue corresponding to X367 is aspartic or glutamic acid, particularly aspartic acid; and residue corresponding to X414 is threonine, asparagine, or glutamine, particularly asparagine.

In some embodiments, the polynucleotides encode an improved amidase polypeptide comprising an amino acid sequence selected from SEQ ID NO: 6, 8, 10, 12, 14, 16, 18, 20, and 22.

In some embodiments, the polynucleotides encoding the engineered amidases are selected from SEQ ID NO: 5, 7, 9, 11, 13, 15, 17, 19, and 21. In some embodiments, the polynucleotides are capable of hybridizing under highly stringent conditions to a polynucleotide selected from SEQ ID NO: 5, 7, 9, 11, 13, 15, 17, 19, and 21, or a complement thereof, where the polynucleotide that hybridizes under highly stringent conditions encode a functional amidase capable of converting the substrate of structural formula (I) to the product of structural formula (II).

In some embodiments, the polynucleotides encode the polypeptides described herein but have about 80% or more sequence identity, about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered amidase. In some embodiments, the reference polynucleotide is selected from polynucleotide sequences represented by SEQ ID NO: 5, 7, 9, 11, 13, 15, 17, 19, and 21.

An isolated polynucleotide encoding an improved amidase polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3^{rd} Ed., Cold Spring Harbor Laboratory Press; and Current Protocols in Molecular Biology, Ausubel. F. ed., Greene Pub. Associates, 1998, updates to 2006, which is incorporated herein by reference.

For bacterial host cells, suitable promoters for directing transcription of the nucleic acid constructs of the present disclosure, include the promoters obtained from the E. coli lac operon, trp operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (VIIIa-Kamaroff et al., 1978, Proc. Natl. Acad. Sci. USA 75: 3727-3731, which is incorporated herein by reference), as well as the tac promoter (DeBoer et al., 1983, Proc. Natl. Acad. Sci. USA 80: 21-25, which is incorporated herein by reference). In some embodiments, the promoters can be those based on bacteriophage promoters, such as phage A promoters.

For filamentous fungal host cells, suitable promoters for directing the transcription of the nucleic acid constructs of the present disclosure include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787, which is incorporated herein by reference), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters can be from 4 the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8:423-488, which is incorporated herein by reference.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra, which is incorporated herein by reference.

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are described by Guo and Sherman, 1995, Mol Cell Bio 15:5983-5990, which is incorporated herein by reference.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region.

Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiol Rev 57:109-137, which is incorporated herein by reference.

Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells can be from the genes for Saccharomyces cerevisiae alpha-factor and Saccharomyces cerevisiae invertase. Other useful signal peptide coding regions are described by Romanos et al., 1992, supra, which is incorporated herein by reference.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (see, for example, WO 95/33836, which is incorporated herein by reference).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the amidase polypeptide of the present invention would be operably linked with the regulatory sequence.

Thus, in another embodiment, the present disclosure is also directed to a recombinant expression vector comprising a polynucleotide encoding an engineered amidase polypeptide, and one or more expression regulating regions such as a promoter and a terminator, a replication origin, etc., depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(S) into which it has been integrated. Furthermore, a single vector or plasmid or two or more vectors or plasmids which together contain the total DNA to be introduced into the genome of the host cell, or a transposon may be used.

The expression vector of the present invention preferably contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol (Example 1) or tetracycline resistance. Suitable markers for yeast host cells are ADE2, HIS3, LEU2, LYS2, MET3, TRP1, and URA3.

Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

The expression vectors of the present disclosure can contain an element(s) that permits integration of the vector into the host cell's genome or autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A on or the origins of replication of plasmids pBR322, pUC19, pACYCI77 (which plasmid has the P15A ori), or pACYC184 permitting replication in E. coli, and pUB110, pE194, pTA1060, or pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes it's functioning temperature-sensitive in the host cell (see, e.g., Ehrlich, 1978, Proc Natl Acad Sci. USA 75:1433, which is incorporated herein by reference).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present invention are commercially available. Suitable commercial expression vectors include p3xFLAG™™ expression vectors from Sigma-Aldrich Chemicals, St. Louis Mo., which includes a CMV promoter and hGH polyadenylation site for expression in mammalian host cells and a pBR322 origin of replication and ampicillin resistance markers for amplification in E. coli. Other suitable expression vectors are pBluescriptll SK(−) and pBK-CMV, which are commercially available from Stratagene, LaJolla Calif., and plasmids which are derived from pBR322 (Gibco BRL), pUC (Gibco BRL), pREP4, pCEP4 (Invitrogen) or pPoly (Lathe et al., 1987, Gene 57:193-201).

### Host Cells

In another aspect, the present disclosure provides a host cell comprising a polynucleotide encoding an improved amidase polypeptide of the present disclosure, the polynucleotide being operatively linked to one or more control sequences for expression of the amidase enzyme in the host cell. Host cells for use in expressing the amidase polypeptides encoded by the expression vectors of the present disclosure are well known in the art and include but are not limited to, bacterial cells, such as E. coli, Lactobacillus, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Appropriate culture mediums and growth conditions for the above-described host cells are well known in the art.

Polynucleotides for expression of the amidase may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

An exemplary host cell is Escherichia coli W3110. The expression vector was created by operatively linking a polynucleotide encoding an improved amidase into the plasmid pCK110900 operatively linked to the lac promoter under control of the lacI repressor (see the vector depicted as FIG. 3 in U.S. Patent Application Publication 2006/0195947, which is incorporated herein by reference). The expression vector also contained the P15a origin of replication and the chloramphenicol resistance gene. Cells containing the subject polynucleotide in Escherichia coli W3110 were isolated by subjecting the cells to chloramphenicol selection.

### Methods of Generating Engineered Amidase Polypeptides.

In some embodiments, to make the improved amidase polynucleotides and polypeptides of the present disclosure, the naturally-occurring or wild-type amidase enzyme used as the starting (or “parent”) sequence for engineering is obtained (or derived) from Ochrobactrum anthropi. In some embodiments, the parent polynucleotide sequence is codon optimized to enhance expression of the amidase in a specified host cell.

As an illustration, a parental polynucleotide sequence encoding the wild-type amidase polypeptide of Ochrobactrum anthropi was constructed from oligonucleotides prepared based upon the amidase sequence available in Genbank database (see, Genbank accession no. Q9ZBA9.3 GI:75475218; see also, Asano et al., 1992, Biochemistry 31(8):2316-2328, incorporated herein by reference). The parental polynucleotide sequence was codon optimized for expression in E. coli and the codon-optimized polynucleotide cloned into an expression vector. Clones expressing the active amidase in E. coli were identified and the genes sequenced to confirm their identity. This codon-optimized polynucleotide sequence (herein designated SEQ ID NO:1) was the parent sequence encoding the wild-type amidase polypeptide from Ochrobactrum anthropi (herein designated SEQ ID NO:2) and was utilized as the starting point for most experiments and library construction of engineered amidases

The engineered amidases can be obtained by subjecting the polynucleotide encoding a naturally occurring amidase to mutagenesis and/or directed evolution methods, as discussed herein and known in the art. An exemplary directed evolution technique useful to make the engineered amidases of the present disclosure is mutagenesis and/or DNA shuffling as described in Stemmer et al., 1994, Proc Natl Acad Sci USA 91:10747-10751; WO 95/22625; WO 97/0078; WO 97/35966; WO 98/27230; WO 00/42651; WO 01/75767 and U.S. Pat. No. 6,537,746, each of which is hereby incorporated by reference herein. Other directed evolution procedures that can be used include, among others, staggered extension process (StEP), in vitro recombination (Zhao et al., 1998, Nat. Biotechnol. 16:258-261), mutagenic PCR (Caldwell et al., 1994, PCR Methods Appl. 3:S136-S140), and cassette mutagenesis (Black et al., 1996, Proc Natl Acad Sci USA 93:3525-3529).

The clones obtained following mutagenesis treatment are screened for engineered amidases having a desired improved enzyme property. Measuring enzyme activity from the expression libraries can be performed using the standard biochemistry technique of monitoring the rate of decrease of substrate and/or increase in product (see, e.g., Examples). Where the improved enzyme property desired is thermal stability, enzyme activity may be measured after subjecting the enzyme preparations to a defined temperature and measuring the amount of enzyme activity remaining after heat treatments. Clones containing a polynucleotide encoding an amidase are then isolated, sequenced to identify the nucleotide sequence changes (if any), and used to express the enzyme in a host cell.

Where the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the invention can be prepared by chemical synthesis using, e.g., the classical phosphoramidite method described by Beaucage et al., 1981, Tet Lett 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources.

In a further embodiment, the present disclosure provides a method of making an amidase polypeptide of the present invention, the method comprising providing a host cell transformed with any one of the described polynucleotides encoding an amidase polypeptide of the present invention; culturing the transformed host cell in a culture medium under conditions that cause said polynucleotide to express the encoded amidase polypeptide; and optionally recovering or isolating the expressed amidase polypeptide. The present invention further provides a method of making a amidase polypeptide, said method comprising cultivating a host cell transformed with a polynucleotide encoding an amidase polypeptide of the present invention under conditions suitable for the production of the amidase polypeptide and recovering the amidase polypeptide.

Typically, recovery or isolation of the amidase polypeptide is from the host cell culture medium, the host cell or both, using protein recovery techniques that are well known in the art, including those described herein.

Following transformation of a suitable host strain and growth (cultivating or culturing) of the host strain to an appropriate cell density, the selected promoter may be induced by appropriate means (e.g., temperature shift or chemical induction) and cells are cultured for an additional period. Cells are typically harvested by centrifugation, disrupted by physical or chemical means, and the resulting crude extract may be retained for further purification. Microbial cells employed in expression of proteins can be disrupted by any convenient method, including freeze-thaw cycling, sonication, mechanical disruption, or use of cell lysing agents, or other methods, which are well known to those skilled in the art.

Many references are available for the culture and production of many cells, including cells of bacterial, plant, animal (especially mammalian) and archebacterial origin. See e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelli, et al., (1989) In vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); Jones, ed. (1984) Plant Gene Transfer and Expression Protocols, Humana Press, Totowa, N.J. and Plant Molecular Biology (1993) R.R.D.Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue (1998) from Sigma-Aldrich, Inc (St Louis, Mo.) (“Sigma-LSRCCC”) and, for example, The Plant Culture Catalogue and supplement (1997) also from Sigma-Aldrich, Inc (St Louis, Mo.) (“Sigma-PCCS”), all of which are incorporated herein by reference.

Engineered amidase enzymes expressed in a host cell can be recovered from the cells and or the culture medium using any one or more of the well known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B™ from Sigma-Aldrich of St. Louis Mo.

Chromatographic techniques for isolation of the amidase polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved amidase enzymes. For affinity chromatography purification, any antibody which specifically binds the amidase polypeptide may be used. For the production of antibodies, various host animals, including but not limited to rabbits, mice, rats, etc., may be immunized by injection with an amidase polypeptide. The polypeptide may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette Guerin) and Corynebacterium parvum.

### Methods and Compounds for Preparation of (S)-Amino Acid Amides

In one aspect, amidases, including the engineered amidase polypeptides described

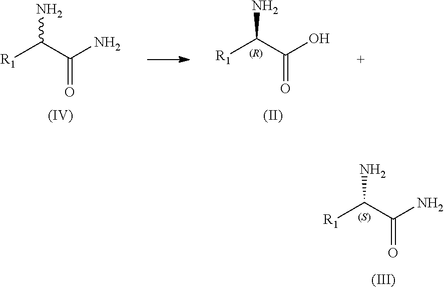

wherein R₁ is a (C₁-C₄) alkyl. In some embodiments, R₁ is an ethyl. The reaction catalyzed by the amidase allows separation of the (S)-amino acid amides from the (R)-amino acid amides. Thus, in some embodiments, the method can comprise contacting a substrate mixture of (IV), e.g., a racemic mixture, with an amidase of the present invention under suitable reaction conditions to form the compound of (II) with the compound of (III) remaining unconverted by the enzyme.

In some embodiments, the method can comprise contacting a mixture of (R)- and (S)-2-aminobutyramide, such as a racemic mixture of (VIII) with a polypeptide of the present invention having amidase activity under suitable reaction conditions for chiral resolution of the (S)-2-aminobutyramide (VII) from the (R)-2-aminobutyramide:

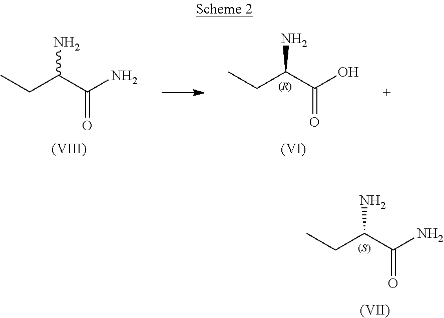

The reactions in Schemes 1 and 2 can be used to prepare (S)-amino acid amides, and specifically (S)-2-aminobutyramide (VII) of high stereometric purity. Suitable reaction conditions for carrying out the above-described reactions are described hereinbelow.

In some embodiments, the reaction conditions in the method comprises a pH of about 6.5 to about 8.0 and a temperature of about 20° C. to about 40° C. In some embodiments the reaction condition is a pH of about 7.5 and a temperature of about 35° C. In some embodiments, the reaction condition is a pH of about 9.5 and a temperature of about 25° C.

In some embodiments of the method, the (S)-2-aminobutyramide remaining is in enantiomeric excess of at least 99%, 99.1%, 99.2%, 99.3%, 99.4%, 99.5%, 99.6%, 99.7%, 99.8%, or 99.9%, or more over the (R)-2-aminobutyramide (which is converted to (R)-2-aminobutyrate).

In some embodiments, the method can comprise contacting a mixture (R)-2-aminobutyramide and (S)-2-aminobutyramide, e.g., a racemic mixture, with an amidase under suitable reaction conditions to convert (R)-2-aminobutyramide to form (R)-2-aminobutyrate, where the reaction condition comprises a pH of about 7.5, a temperature of about 35° C., about 475 g/L of 2-aminobutyramide substrate, and about 1 g/L of engineered amidase (or naturally occurring amidase) polypeptide in a reaction time of about 8 hrs, wherein at least 40%, 45%, 46%, 47%, 48% or 49% of the racemic substrate is converted to (R)-2-aminobutyrate.

In some embodiments, the method can comprise contacting a mixture (R)-2-aminobutyramide and (S)-2-aminobutyramide, e.g., a racemic mixture, with an amidase under suitable reaction conditions to convert (R)-2-aminobutyramide to form (R)-2-aminobutyrate, where the reaction condition comprises a pH of about 9.5, a temperature of about 25° C., about 300 g/L of 2-aminobutyramide substrate in a reaction time of about 20 hrs, wherein at least 40%, 45%, 46%, 47%, 48% or 49% of racemic substrate is converted to (R)-2-aminobutyrate.

In some embodiments, the biocatalytic process for preparing chirally pure (S)-2-aminobutyramide can be carried out wherein the (S)-2-aminobutyramide is separated from the (R)-2-aminobutyrate conversion product by extraction with a polar organic solvent. In some embodiments, the polar organic solvent is selected from a (C₁-C₄) alcohol (e.g., methanol, ethanol, butanol, isopropanol, isobutanol, and tert-butanol), acetone, methylethylketone, acetonitrile, or mixtures thereof. The efficiency of the polar organic solvent extraction step for isolating (S)-2-aminobutyramide from the aqueous biocatalytic reaction mixture is surprising in view of the recognized water miscibility of these solvents (e.g., isopropanol). The extraction can be carried out in a step-wise or continuous manner (see e.g., Examples 11 and 12).

In some embodiments, the extraction is carried out in a step-wise manner wherein: (1) organic solvent is added to the aqueous biocatalytic reaction mixture following completion of the biocatalytic conversion step; (2) the mixture is heated; (3) the organic layer is withdrawn and retained; and (4) these steps (1)-(3) are repeated at least once more; whereby the desired (S)-2-aminobutyramide product is extracted into the organic layer. In some embodiments, the pH of the aqueous layer can be adjusted to alkaline pH (e.g., about pH 7.5-9.5) following prior to each step of adding the organic solvent. Such pH adjustment facilitates extraction efficiency when e.g., lower carboxylic acid salts or sulfate salts of 2-aminobutyramide are utilized in the biocatalytic conversion process, as described in further detail below.

In some embodiments, the method comprises using a lower carboxylic acid salt or sulfate salt of the 2-aminobutyramide (“2-ABM”) in the biocatalytic reaction, wherein the lower carboxylic acid salt or sulfate salt includes, but is not limited to formate, acetate, propionate, and particularly the acetate salts, and sulfate salts. In some embodiments, the use of a lower carboxylic acid salt of the 2-ABM results in greater percentage conversion of 2-ABM and a greater yield of the chirally resolved product.

Additionally, in some embodiments, the use of a lower carboxylic acid salt of the 2-ABM results in a decreased amount of byproducts in the amidase catalyzed conversion reaction. For example, as shown by the results listed in Tables 3 and 4, the use of halide salts of 2-ABM in the amidase reaction can result in decreased percent conversion and increased percent byproducts at higher substrate loading conditions, e.g., at substrate levels equivalent to a freebase form concentration of 300 g/L or higher.

TABLE 3

| | | | Equivalent | | |
|---|---|---|---|---|---|
| Enzyme | 2-ABM | Salt form | conc. of | rac-2-ABM | |
| (SEQ ID | salt | conc. used | freebase | % conversion | % |
| NO) | form | (g/L) | (g/L) | in 24 h | byproduct |
| 16 | HBr | 350 | 200 | 100 | 0.8 |
| 16 | HBr | 540 | 300 | 76 | 3.5 |
| 16 | acetate | 475 | 300 | 100 | 1.6 |
| 16 | acetate | 550 | 350 | 100 | 1.8 |
| 8 | HCl | 400 | 300 | 74 | nd |

TABLE 4

| | | | Equivalent | |
|---|---|---|---|---|
| Enzyme | 2-ABM | Salt form | conc. of | rac-2-ABM |
| (SEQ ID | salt | conc. used | free base | % conversion |
| NO) | form | (g/L) | (g/L) | in 5 h |
| 2 (WT) | HCl | 475 | 350 | 86.1 |
| 2 (WT) | H₂SO₄ | 530 | 350 | 95.4 |
| 2 (WT) | HOAc | 560 | 350 | 94.7 |
| 8 | HCl | 475 | 350 | 93.7 |
| 8 | H₂SO₄ | 530 | 350 | 97.0 |
| 8 | HOAc | 560 | 350 | 97.4 |

NOTE:
Salts prepared in situ: HCl, HOAc, H₂SO₄. Reactions were prepared utilizing 2-ABM freebase solution (pH 9.3) and adjusting to pH 7.5 by adding the appropriate acid before the enzyme was added. Therefore, the reacting substrate was present as the indicated salt.

Accordingly, in some embodiments, useful for the methods herein are compositions of lower carboxylic acid salts or sulfate salts of the amino acid amides. In some embodiments, the lower carboxylic acid salts are selected from formate, acetate and propionate salts of the amino acid amide. In some embodiments, the composition can comprise a lower carboxylic acid or sulfate salts of the amino acid amides represented by the structural formulas (Ia) and (IIIa) as shown below:

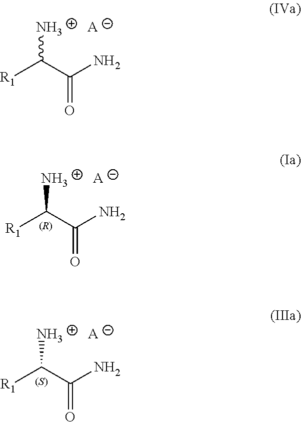

In some embodiments, the composition is a formate, acetate, propionate or sulfate salt of a racemic mixture as represented by the structural formula (IVa).

In some embodiments, the composition can comprise a mixture of the lower carboxylic acid salts or sulfate salts of the (R)-amino acid compound of formula (II) and the (S)-amino acid amide compound of formula (III), where R₁ can be a lower alkyl, i.e., a (C₁-C₄) alkyl.

In some embodiments, useful for the methods herein are compositions of lower carboxylic acid salts or sulfate salts of the (R)-2-aminobutyramide or (S)-2-aminobutyramide. In some embodiments, the composition can comprise a lower carboxylic acid salts or sulfate salts of a mixture of (R) 2-aminobutyramide and (S) 2-aminobutyramide, such as a lower carboxylic acid salt or sulfate salt of racemic 2-aminobutyramide.

In some embodiments, the lower carboxylic acid salts and sulfate salts of the substrates can be used in methods for the preparation of (S)-amino acid amides, where the method comprises contacting a substrate mixture of the compound of structural formula (II) and (III) in the form of lower carboxylic acid salts, with an amidase, thereby converting the mixture to (R) amino acid and (S) amino acid amides. In some embodiments of the method, the substrate mixture can be a racemic mixture. In some embodiments, the substrate mixture is a mixture of the lower carboxylic acid salts of (R) and (S)-2-aminobutyramide.

In some embodiments, the amidases useful in the reaction with the lower carboxylic acid salts of the substrate can have an amino acid sequence that is at least 80%, 85%, 90%, 91%, 92% 93%, 94%, 95%, 96%, 97%, 98%, or at least 99% or more identical to the amidase of SEQ ID NO:2. Exemplary amidases include, by way of example and not limitation, the polypeptides corresponding to S02709264 (SEQ ID NO: 2); gi|75475218|sp|Q9ZBA9.3|DAP_OC (SEQ ID NO: 23); gi|53008743|ref|YP_{—}001369958 (SEQ ID NO: 24); gi|23500673|ref|NP_{—}700113.1| (SEQ ID NO: 25); gi|17988695|ref|NP_{—}541328.1| (SEQ ID NO: 26); gi|48558345|ref|YP_{—}001257867 (SEQ ID NO: 27); gi|126464718|ref|YP_{—}001045831 (SEQ ID NO: 28); gi|77465254|ref|YP_{—}354757.1| (SEQ ID NO: 29); gi|169785843|ref|XP_{—}001827382 (SEQ ID NO: 30); gi|58039691|ref|YP_{—}191655.1 (SEQ ID NO: 31); gi|114762789|ref|ZP_{—}01442223.1 (SEQ ID NO: 32); gi|46126321|ref|XP_{—}387714.1| (SEQ ID NO: 33); gi|76057819|emb|CAH19237.1|_p (SEQ ID NO: 34); gi|145241538|ref|XP_{—}001393415 (SEQ ID NO: 35); and variants thereof. Suitable variants of these amidases include those having any of the sequence features of the invention amidase polypeptides described herein.

Generally, the amidase substrates of racemic structural formula (IV) can be obtained by standard chemistries. For instance, racemic 2-aminobutyramide can be prepared from propanal (XIII) which is converted by a Strecker reaction to the 2-aminobutanenitrile (XIV). Hydration of the 2-aminobutanenitrile with a base in presence of acetone results in racemic 2-aminobutyramide (XV), which can be isolated as a freebase by extraction of the salted aqueous solution with isopropanol. See, e.g., U.S. Pat. No. 4,243,814, which is hereby incorporated by reference herein. The freebase is readily converted to the acetate salt by the addition of acetic acid in isopropanol (Scheme 3):

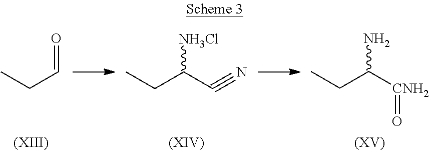

In some embodiments, the (S)-2-aminobutyramide can be used for the synthesis of levetiracetam, (2S)-2-(2-oxopyrrolidin-1-yl)butanamide, having the following structural formula (X):

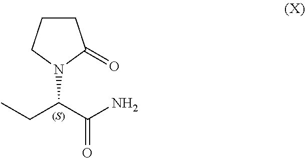

The synthesis of levetiracetam from (S)-2-aminobutyramide can use the process described in published patent publication EP 1 566 376 (which is hereby incorporated by reference herein), as illustrated in Scheme 4:

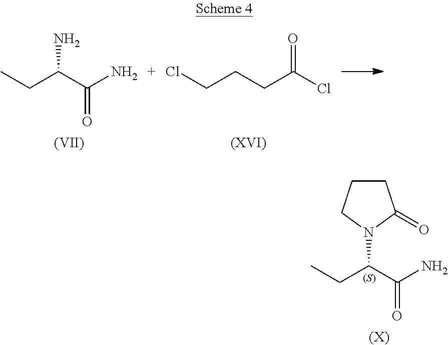

Accordingly, in a method for the synthesis of levetiracetam, a step in the method can comprise contacting a mixture of (R)-2-aminobutyramide and (S)-2-aminobutyramide (VII), such as in the form of lower carboxylic acid salts or sulfate salts, with an amidase, such as an engineered amidase of the present invention, to form (S)-2-aminobutyramide in enantiomeric excess of the (R)-2-aminobutyramide.

In some embodiments, levetiracetam is synthesized using the process illustrated in Scheme 5 (below):

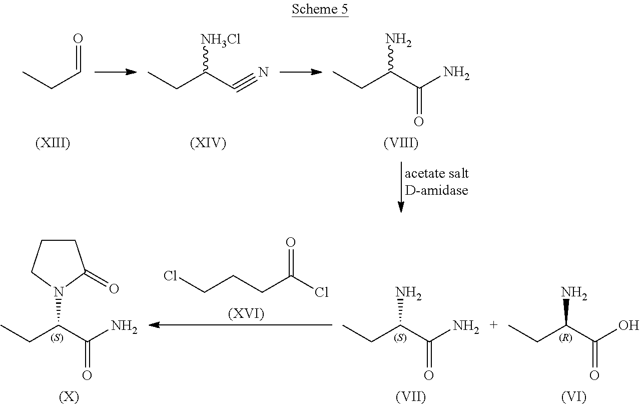

The process illustrated in Scheme 5 comprises: (a) converting propanal (XIII) to a 2-aminobutanenitrile (XIV); (b) reacting the 2-aminobutanenitrile with base to form racemic 2-aminobutyramide (VIII); (c) forming an acetate salt of the racemic 2-aminobutyramide and contacting the racemic mixture with an amidase to form an enantiomeric excess of (S)-2-aminobutyramide (VII); (d) reacting the (S)-2-aminobutyramide (VII) with 4-chlorobutyryl chloride (XVI) to form levetiracetam, (2S)-2-(2-oxopyrrolidin-1-yl)butanamide (X). The present invention provides a method for making (2S)-2-(2-oxopyrrolidin-1-yl)butanamide (Levetiracetam), said method comprising: (a) contacting a racemic mixture of 2-aminobutyramide (VIII) or salt thereof with an engineered amidase of the present invention, thereby forming an enantiomeric excess of (S)-2-aminobutyramide (VII); (b) contacting the (s)-aminobutyramide (VII) with 4-chlorobutyryl chloride (XVI) under reaction conditions suitable for forming (2S)-2-(2-oxopyrrolidin-1-yl)butanamide (X) (Levetiracetam); and (c) optionally isolating or purifying the (2S)-2-(2-oxopyrrolidin-1-yl)butanamide (X). Reaction conditions suitable for forming (2S)-2-(2-oxopyrrolidin-1-yl)butanamide (X) from 4-chlorobutyryl chloride (XVI) and (2S)-aminobutyramide (VIII) and methods for purifying levetiracetam are well known in the art and are described, for example, in EP 1 566 376 A1, which is incorporated herein by reference. The present invention includes (2S)-2-(2-oxopyrrolidin-1-yl)butanamide (Levitiracetam) made by the processes described herein.

In some embodiments, the amidases can be used in a method to synthesize analogs of leviteracetam. In some embodiments, the amidases can be used in a method to synthesize analogs brivaracetam and seletracetam.

In some embodiments, the amidases can be used in a method for preparing brivaracetam, (2S)-2-[(4R)-2-oxo-4-propylpyrrolidin-1-yl]butanamide, having the following structural formula (XI):

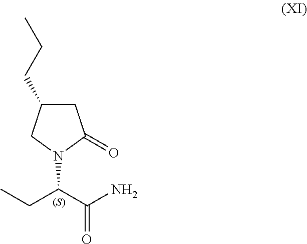

Accordingly, in a method for the synthesis of brivaracetam (XI), a step in the method can comprise contacting a mixture of (R)-2-aminobutyramide and (S)-2-aminobutyramide, such as in the form of lower carboxylic acid salts or sulfate salts, with an amidase to form (S)-2-aminobutyramide (VII) in enantiomeric excess of the (R)-2-aminobutyramide. In some embodiments, the amidase is an engineered amidase described herein. Methods for preparing brivaracetam (XI) from (S)-2-aminobutyramide (VII) are known in the art. See, for example, WO 2007/065623, which is incorporated herein by reference.

The present invention provides a method for preparing (2S)-2[(4R)-2-oxo-4-propylpyrrolidin-1-yl]butanamide (XI) (brivaracetam), the method comprising: (a) providing a racemic mixture of (S)-2-aminobutyramide and (R)-2-aminobutyramide or salts thereof; (b) contacting the racemic mixture (S)-2-aminobutyramide and (R)-2-aminobutyramide (or salts thereof) with an amidase polypeptide of the present invention under reaction conditions suitable to convert (R)-aminobutyramide to (R)-2-aminobutyrate, thereby forming (S)-2-aminobutyramide in stereomeric excess of (R)-2-aminobutyramide; (c) contacting the (S)-2-aminobutyramide with (S)-6,6-Dimethyl-1-propyl-5,7-dioxa-spiro[2.5]octane-4,8-dione (compound (XIa)),

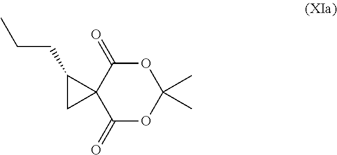

under conditions suitable for forming products, (R)-1-((S)-1-carbamoyl-propyl)-2-oxo-4-propyl-pyrrolidine-3-carboxylic acid (XIb) and 1-((S)-1-carbamoyl-propyl)-2-oxo-5-propyl-pyrrolidine-3-carboxylic acid (XIc):

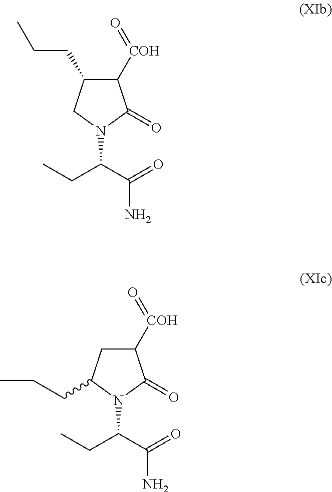

(d) decarboxylating products (XIb) and (XIc) under reaction conditions suitable to form compounds (XI) and (XIe):

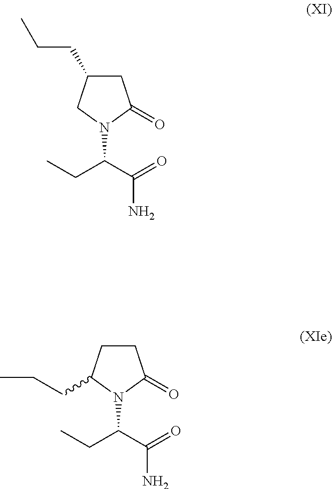

Reaction conditions for converting compounds (XIa) and (S)-2-aminobutyramide (VII) to compounds (XIb) and (XIc) are well known in the art and include carrying out the reaction in a solvent, such as, for example, acetonitrile, and refluxing for 10 hours. See, for example, WO 2007/065634, which is incorporated herein by reference. Decarboxylation of products (XIb) and (XIc) to form compounds (XI) and (XIe) may be carried out at atmospheric pressure in the presence of a solvent having a boiling point greater than 110° C. (e.g., toluene, dimethylformamide, dimethylsulfoxide, N-methyl-2-pyrrolidone, methylsiobutylketone, and the like). The reaction may be carried out at a temperature in the range of from about 110° C. to about 130° C., such as, for example 120° C. Methods for carrying out the decarboxylation step are described in the art. See, for example, PCT publication WO 2007/065634 and A. P. Krapcho et al., Tetrahedron Lett. (1967) 8:215-217, both of which are incorporated herein by reference.

In some embodiments, the amidases can be used in a method for preparing seletracetam, (2S)-2-[(4R)-4-(2,2-difluoroethenyl)-2-oxo-pyrrolidin-1-yl]butanamide, having the following structural formula (XII):

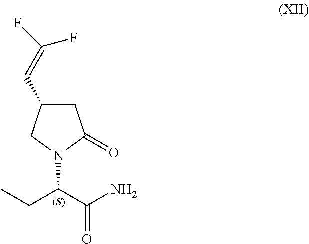

Accordingly, in a method for the synthesis of seletracetam (XII), a step in the method can comprise contacting a mixture of (R)-2-aminobutyramide and (S)-2-aminobutyramide, such as in the form of lower carboxylic acid salts or sulfate salts, with an amidase to form (S)-2-aminobutyramide in enantiomeric excess of the (R)-2-aminobutyramide. In some embodiments, the amidase is an engineered amidase described herein.

More specifically, the present invention provides a method for preparing seletracetam ((S)-2-[(4R)-4-(2,2-difluoroethenyl)-2-oxo-pyrrolidin-1-yl]butanamide), the method comprising: (a) providing a racemic mixture of (S)-2-aminobutyramide and (R)-2-aminobutyramide or salts thereof; (b) contacting the racemic mixture of (S)-2-aminobutyramide and (R)-2-aminobutyramide (or salts thereof) with an amidase polypeptide of the present invention under reaction conditions suitable to convert (R)-aminobutyramide to (R)-2-aminobutyrate, thereby forming (S)-2-aminobutyramide in stereomeric excess of (R)-2-aminobutyramide; (c) contacting the (S)-2-aminobutyramide with the compound having formula (XIIa),

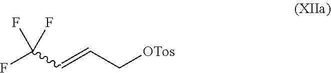

under reaction conditions suitable for forming product (XIIb),

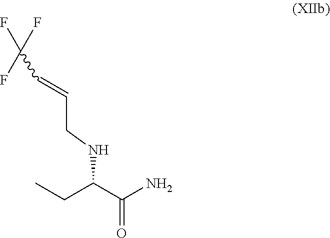

(d) contacting compound (XIIb) with dimethyl malonate (XIIc),

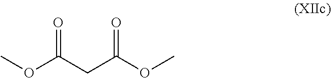

under reaction conditions suitable to form compound (XIId):

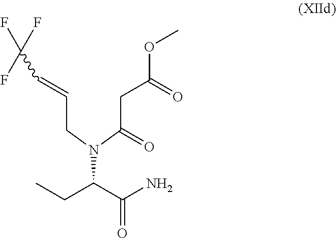

(e) cyclizing compound (XIId) to under reaction conditions suitable to form compound (XIIe):

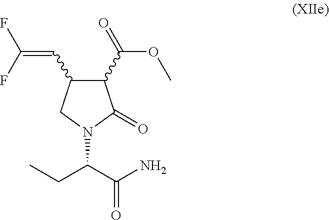

(f) hydrolyzing compound (XIIe) under reaction conditions suitable to form compound (XIIf):

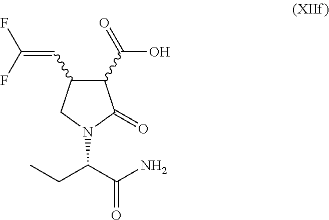

(g) recrystallizing compound (XIIf) to form compound (XIIg),

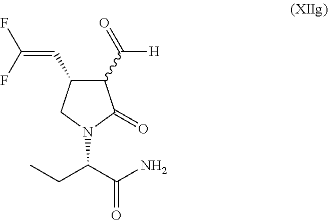

and (f) decarboxylating compound (XIIf) under reaction conditions suitable to form compound (XII),

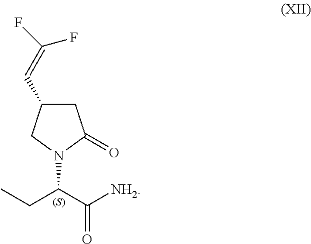

Reaction conditions suitable for carrying out the above specified steps are known in the art, and are specifically described, for example, in WO 2005/121082, which is incorporated herein by reference.

Various features and embodiments of the disclosure are illustrated in the following representative examples, which are intended to be illustrative, and not limiting.

### EXAMPLES

### Example 1

### Construction of D-Aminopeptidase Expression Vectors

The D-Aminopeptidase encoding (DAP) gene was designed for expression in E. coli using standard codon optimization based on the reported amino acid sequence of the Ochrobactrum anthropi D-Aminopeptidase (SEQ ID NO: 2) (Codon-optimization software is reviewed in e.g., “OPTIMIZER: a web server for optimizing the codon usage of DNA sequences,” Puigbó et al., Nucleic Acids Res. 2007 July; 35 (Web Server issue): W126-31. Epub 2007 Apr. 16.) Genes were synthesized using oligonucleotides composed of 42 nucleotides and cloned into expression vector pCK110900 (vector depicted as FIG. 3. 3 in US Patent Application Publication 20060195947, which is hereby incorporated by reference herein) under the control of a lac promoter. The expression vector also contained the P15a origin of replication and the chloramphenicol resistance gene. Resulting plasmids were transformed into E. coli W3110 (fhu-) using standard methods. Two rounds of directed evolution of the codon-optimized DAP gene was carried out yielding the variant sequences listed in Table 2. Briefly, an E315G mutation found in the wild-type Dap backbone was removed using site-directed mutagenesis and a splicing-overlap extension (SOE) approach. A series of random mutagenesis libraries were prepared. From these libraries, a variant containing the mutations G317D, G367D, S414N was identified having 1.2-fold better activity than wild-type, and was used as the polypeptide backbone to prepare the next library of mutations. The next library was prepare where the amino acids at the X290 position of the Dap were varied. From this X290 library, two variants (A2905 and A290T) produced ˜5% byproduct in the desired 2-ABM reactions (see below), which was improved over the parent polypeptide (˜10% byproduct). Further, A290S displayed 1.5-fold improved conversion when compared with the parent (see FIG. 2).

### Example 2

### Shake Flask Procedure for Production of D-Aminopeptidase Powders

A single microbial colony of transformed E. coli containing a plasmid with the DAP gene of interest was inoculated into 50 mL Luria Bertani broth containing 30 μg/mL chloramphenicol and 1% glucose. Cells were grown overnight (at least 16 hrs) in an incubator at 30° C. with shaking at 250 rpm. The culture was diluted into 250 mL Terrific Broth (12 g/L bactotryptone, 24 g/L yeast extract, 4 mL/L glycerol, 50 mM potassium phosphate, pH 7.0, 1 mM MgSO₄, 30 μg/mL chloramphenicol) in 1 liter flask) to an optical density at 600 nm (OD600) of 0.2 and allowed to grow at 30° C. Expression of the DAP gene was induced with 1 mM IPTG (isopropyl β-D-1-thiogalactopyranoside) when the OD600 of the culture was 0.5 to 0.6 and incubated overnight (at least 16 hrs). Cells were harvested by centrifugation (5000 rpm, 15 min, 4° C.) and the supernatant discarded. The cell pellet was suspended with an equal volume of cold (4° C.) 100 mM triethanolamine (chloride) buffer, pH 7.0 (including 2 mM MgSO₄), and harvested by centrifugation as above. The washed cells were resuspended in two volumes of the cold triethanolamine (chloride) buffer and passed through a Cell Disruptor once at 16500 psi while maintained at 4° C. Cell debris was removed by centrifugation (10000 rpm, 30 min, 4° C.). The clear lysate supernatant was collected and stored at −20° C. Lyophilization of frozen clear lysate provided a dry powder of crude D-aminopeptidase enzyme (Dap).

### Example 3

### Analytical Methods for Determining Conversion of Racemic-2-Aminobutyramide (2-ABM) and the Resulting Enantiomeric Excess of (S)-2-ABM

O-phthalaldehyde (OPA) derivatization method to detect the substrates and products: An automated OPA derivatization method was developed for UV detection of the amine-containing substrates racemic 2-aminobutyramide (rac-2-ABM) and the product 2-aminobutyric acid (2-ABA). See e.g., FIGS. 3A and 3B. Use of the OPA reagent with N-acetylcysteine affords a diastereomeric derivative that can be analyzed on a standard C18 HPLC column. The method was found to be linear between 0.1 and 50 mM when 20 μL of sample solution is added to 100 μL of the OPA reagent.

OPA reagent preparation: 420 mg OPA solid was dissolved in 10 mL of ethanol, and the solution was mixed with 510 mg N-Acetyl-L-cysteine in 90 mL of 0.1 M Sodium Tetraborate (pH 10).

Chiral HPLC to determine the conversion of rac-ABM and enantiomeric excess of S-2-ABM: Conversion of rac-2-ABM to R-2-ABA and the abundance of the R and S enantiomers of 2-ABM following conversion were determined using an Agilent HPLC 1200 equipped with a SYNERGI® 4u Fusion-RP 80A column (4.6×50 mm and 12.5 mm guard column) (Phenomenex, Torrance, Calif., USA) with acetonitrile/40 mM ammonium acetate pH 5.0 (18/82) as eluent at a flow rate of 1.5 mL/min at 60° C. As illustrated by HPLC chromatogram depicted in FIG. 3B, retention times of the 2-ABA, S-2-ABM and R-2-ABM were approximately 1.0, 2.2, and 2.7 minutes, respectively.

LC/MS/MS to determine stereomeric purity of 2-ABM: Alternatively, the ratio of S to R enantiomers of 2-ABM following enzymatic conversion was determined using a Daicel CR+ column (4.6×150 mm and 12.5 mm guard column) (Chiral Technologies, West Chester, Pa., USA) with 0.5% acetic acid in deionized water as eluent at a flow rate of 0.8 mL/min at 0° C. The retention times of the R-2-ABM and S-2-ABM were 1.4 and 1.5 minutes respectively. A multiple reaction monitoring (MRM) method on the LC/MS was used for monitoring the 2-ABA (mw=103) and the 2-ABM (mw=102).

### Example 4

### Evaluation of Wild-Type Amidase for Hydrolysis of R-2-aminobutyramide

The wild-type Dap enzyme from Ochrobactrum anthropi was evaluated for activity towards rac-ABM. A 1 mL solution of rac-ABM (100 g/L), Dap (1 g/L) in water (pH 8.0) was incubated at 40° C. for 22 hours. LC/MS/MS analysis on the 22 hour reaction mixture has revealed that the overall conversion of rac-ABM was around 50% and the ratio of (S)-2-ABM to (R)-2-ABM was ˜86000/1, which gives S-2-ABM in 99.9% e.e. This example confirmed that wild-type Dap displayed stereoselectivity for conversion of (R)-2-ABM with low substrate loading. The WT Dap was selected as starting point for enzyme evolution towards the catalyst robustness.

### Example 5

### High Throughput HPLC Assay to Determine Amidase Activity and Selectivity

Plasmid libraries containing evolved DAP genes were transformed into E. coli W3110 and plated on Luria-Bertani (LB) agar plates containing 1% glucose and 30 μg/mL chloramphenicol (CAM). After incubation for at least 18 hours at 30° C., colonies were picked using a Q-bot® robotic colony picker (Genetix USA, Inc., Beaverton, Oreg.) into shallow, 96-well well microtiter plates containing 180 μL LB, 1% glucose and 30 μg/mL CAM. Cells were grown overnight at 30° C. with shaking at 200 rpm and 85% humidity. 20 μL of this culture was then transferred into 96-well microtiter deep well plates containing 380 μL 2× yeast tryptone (YT) medium and 30 μg/mL CAM. After incubation of deep-well plates at 30° C. with shaking at 250 rpm for 2 hours (OD₆₀₀ 0.6-0.8), recombinant gene expression by the cell cultures was induced by IPTG to a final concentration of 1 mM. The plates were then incubated at 30° C. with shaking at 250 rpm and 85% humidity for overnight (˜15-18 hours).

Cells were pelleted via centrifugation, resuspended in 200 μL lysis buffer and lysed by shaking at room temperature for 2 hours. The lysis buffer contained 100 mM triethanolamine (chloride) buffer, pH 7.5, 1 mg/mL lysozyme and 500 μg/mL polymixin B sulfate. The plates were centrifuged at 4000 rpm for 15 minutes and the clear supernatant (lysate) used in the HPLC assay.

In deep-well, 96-well microtiter plates 20 μL of clear supernatant was added to 100 μL of an assay mixture (pH 7.5, adjusting with acetic acid) consisting of 300-350 g/L 2-ABM, 5% isopropanol. After sealing with aluminum/polypropylene laminate heat seal tape (Cat# 06643-001; Velocity 11, Menlo Park, Calif.), the plates were incubated at 35° C. for up to 20 hrs. In shallow well microtiter plates, 20 μL of the reaction mixture was diluted 10-fold with 180 μL of water/acetonitrile (90/10). In the same way, the 10-fold diluted sample was diluted another 10-fold, which gave a total 100-fold dilution. Then 20 μL of 100-fold diluted samples were transferred to a new shallow well microtiter plate. These sample plates then were sealed with heat seal tape to prevent evaporation.

OPA reagent was prepared as in Example 3 and 800 μL of OPA reagent was transferred into each well of a new 96-deep-well microtiter plate.

On HPLC, 100 μL of OPA reagent was transferred from the deep-well plates and injected into each well of the shallow-well microtiter plates containing the 100-fold diluted samples. The OPA derivatized samples were analyzed by HPLC by the method of Example 3.

### Example 6

### Hydrolysis of 2-Aminopropionitrile to rac-ABM Freebase

A 1-liter, 3-necked, round-bottomed flask was fitted with a mechanical stirring paddle, pH probe, thermocouple, 60-mL additional funnel, and an ice/NaCl bath. The reactor was charged with the following in order: 2-aminopropionitrile, 61.2 g; water, 200 mL; acetone, 39 mL. The stirring was started at ˜100 RPM. When temperature reached −5° C., NaOH (50% wt/wt, 34 mL) was added dropwise via the additional funnel over approximately 20 min. The reaction was stirred in the ice/NaCl bath for 40 min. Concentrated HCl (13.5 mL) was then added to the reactor to bring pH to 9.5 while maintaining the temperature below 5° C. The ice/NaCl bath was replaced with a heating mantle, and the reactor warmed to 40° C. The reactor was charged with NaCl, (37 g). The aqueous phase was extracted at 60° C. with isopropyl alcohol (2×300 mL) with stirring for at least 30 min. The isopropyl alcohol fractions were combined and concentrated to dryness under reduced pressure. The residue was triturated with isopropyl alcohol (350 mL) and filtered. The filtrate was concentrated to give the crude rac-ABM freebase as a white solid, 49 g (95% theory).

### Example 7

### Conversion of rac-ABM Freebase into its Acetic Acid Salt

A 1-liter, 3-necked, round-bottomed flask equipped with a temperature probe and a mechanical stirrer was charged with rac-ABM free base (54 g) and 2-propanol (450 mL) to make a concentration of about 1.2 M. The clear yellow solution was cooled in an ice water bath to 10° C. Glacial acetic acid was added to the flask in one portion (30 mL). The internal temperature rose to 33.5° C. The rac-ABM acetate salt precipitated as a white solid. The mixture was cooled to 5° C., and the solid was collected by filtration and washed with cold 2-propanol (50 mL). Upon drying under a vacuum at 40° C. overnight, the white solid rac-ABM acetate salt weighed 70.6 g (82.5% theory).

### Example 8

### Determination of Enzymatic Conversion Utilizing HPLC Analysis of the OPA Derivative

A derivatization reagent was prepared by combining the following: 1) 26.8 mg o-phthalaldeyhde in 1 mL ethanol; 2) 8 mL 0.1 M Sodium tetraborate decahydrate in distilled water, pH 10; and 3) 1 mL 1.0 M N-Acetyl-L-cysteine in distilled water. The solution was pale straw colored and used within three days.

A reaction sample was prepared for HPLC analysis through two dilutions. Dilution 1: 0.3 mL of reaction solution was diluted to 4 mL with deionized water; Dilution 2: 15 μL of dilution 1 was diluted to 500 μL with deionized water in an HPLC vial. This sample was treated directly with 90 μL of OPA reagent and 10 μL of the resulting solution was immediately injected on an HPLC.

The following HPLC column parameters were used: Column: Agilent XDB-C18 (5 μm, 4.6×150 mm); Column temperature: 40° C.; Detection wavelength: 336 nm (only derivatized peaks are seen at this wavelength); Flow rate: 1.8 mL/min; Mobile phase: C=acetonitrile, D=100 millimolar ammonium acetate in water; Timetable: 0 to 10.45 min (15:85, C:D), 10.50 min (24:76, C:D), 13.0 min (24:76, C:D), 13.05 min (15:85, C:D), 18 min (15:85, C:D). Retention times of the derivatized compounds were as follows: (S)-2-ABA: 3.0 min; (R)-2-ABA: 3.1 min; (S)-2-ABM: 8.9 min; (R)-2-ABM: 10.9 min.

### Example 9

### Biocatalytic Conversion of rac-ABM Acetic Acid Salt into (S)-2-ABM and (R)-2-ABA

A 0.25-liter, 3-necked, round-bottomed jacketed flask was fitted with a magnetic stir bar, a magnetic stirrer, a pH probe, a thermocouple, and a temperature-controlled recirculation bath. The flask was charged with the following in order: rac-ABM acetic acid salt (59.0 g) and deionized water (69 mL). The mixture was stirred to give a clear solution with pH=6.9. With cooling the reactor is charged with 2.2 g of 50% aqueous NaOH to bring the pH to 7.5. The reactor was stabilized at 25° C. with slow stirring. The reactor was charged with 120 mg of the Dap variant polypeptide (SEQ ID NO:16) dissolved in deionized water (0.6 mL). The enzymatic conversion was allowed to stir at 25° C. with periodic monitoring by HPLC as described in Example 8. The reaction was complete in 18 h. The reaction solution was adjusted to pH 5.9 using glacial acetic acid (4.6 mL). The suspension was cooled to 5° C., aged 30 min, and the solid collected by filtration. The solid was washed with 10 mL of 5° C. water and discarded. The combined filtrate was replaced in the reactor. The pH was adjusted to 9.3 by slowly adding 14.4 g of 50% aqueous NaOH while maintaining the temperature <25° C. The solution was then transferred to an extractor for separation as described in Examples 11 and 12 below.

### Example 10

### Biocatalytic Resolution of rac-ABM Freebase to (S)-2-ABM and (R)-2-ABA

A 0.25-liter, 3-necked, round-bottomed jacketed flask was fitted with a magnetic stir bar, a magnetic stirrer, a pH probe, a thermocouple, and a temperature-controlled recirculation bath. The flask was charged with the following in order: rac-ABM freebase (45.0 g) and deionized water (60 mL). The mixture was stirred to give a clear solution with pH 9.5. With cooling the reactor was charged with 15 mL glacial acetic acid to bring the pH to 7.5. The reactor was stabilized at 25° C. with slow stirring. The reactor was charged with 60 mg Dap variant polypeptide (SEQ ID NO:16) dissolved in deionized water (0.6 mL). The enzymatic reaction was allowed to stir at 25° C. with periodic monitoring of conversion by HPLC. The reaction was complete in 16 h. The reaction solution was adjusted to pH 5.9 using glacial acetic acid (12 mL). The suspension was cooled to 5° C., aged 30 min, and the solid collected by filtration. The solid was washed with 10 mL of 5° C. water and discarded. The combined filtrate was replaced in the reactor. The pH was adjusted to 9.6 by slowly adding 28.3 mL of 50% aqueous NaOH while maintaining the temperature <25° C. The mixture was then transferred to an extractor for separation as described Examples 11 and 12.

### Example 11

### Separation of (S)-2-ABM from (R)-2-ABA Utilizing Stepwise Acetonitrile Extraction

A solution obtained from the biocatalytic conversion described above (60 mL) was transferred to a 0.25-liter, 3-necked, round-bottomed flask fitted with a magnetic stir bar, a magnetic stirrer, a pH probe, a thermocouple, and a temperature-controlled heating mantle. The flask was charged with 120 mL acetonitrile, and the mixture was heated to 70° C. with stirring for 15 min. The upper layer was withdrawn, and the lower aqueous layer was adjusted to pH 9.2 with 4.0 g of 50% aqueous NaOH. The flask was charged with 120 mL acetonitrile and the mixture was heated to 70° C. with stirring for 15 min. The upper organic layer was withdrawn and the lower aqueous layer was adjusted to pH 9.4 with 0.8 g of 50% aqueous NaOH. The flask was charged with 120 mL acetonitrile and the mixture was heated to 70° C. with stirring for 15 min. The upper organic layer was withdrawn. The removed organic layers were combined and concentrated to dryness affording 8.68 g of white solid. This solid was taken into MeOH (80 mL) and the resulting solution transferred to a 0.25 liter, 3-necked round-bottomed jacketed flask fitted with mechanical stirrer, thermocouple, an HCl gas inlet, and an ice bath. The flask was cooled to 10° C. and dry HCl gas was passed into the vessel (subsurface) until the pH=1. The suspension was cooled to 5° C. and the solid was collected by filtration. The solid was dried to constant weight in a vacuum oven at 40° C. affording 6.42 g (50% theory).

### Example 12

### Separation of (S)-2-ABM from (R)-2-ABA Utilizing Vacuum Continuous Extraction

A solution obtained from the biocatalytic conversions described above (120 mL) was transferred to a continuous extractor with acetonitrile (500 mL size from Aldrich; 155 mL in extractor, 205 mL in distillation vessel). Vacuum was applied to 200 torr and continuous extraction was conducted for 21 h. The contents of the distillation vessel were concentrated to dryness and the residual mass was taken up in MeOH (60 mL). The resulting solution was transferred to a 0.25 liter, 3-necked round-bottomed jacketed flask fitted with mechanical stirrer, thermocouple, an HCl gas inlet, and a temperature-controlled recirculation bath. The flask was cooled to 10° C. and dry hydrogen chloride gas was passed into the vessel (subsurface) until the pH=1. The resulting suspension was cooled to 5° C. and the solid collected by filtration. The filter cake was washed with 10 mL cold MeOH. The solid was dried to constant weight in a vacuum oven at 40° C. affording 15.3 g (79.4% theory). The liquors were concentrated to dryness and triturated with isopropyl alcohol. The resulting solid was collected by filtration. The filter cake was washed with 15 mL IPA. The solid was dried to constant weight in a vacuum oven at 40° C. affording 1.55 g (8.1% theory).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(S).

1. An engineered amidase polypeptide capable of stereospecifically converting (R)-2-aminobutyramide to (R) 2-aminobutyric acid with an increased conversion rate as compared to the polypeptide of SEQ ID NO: 2 at pH 7.5 at 35° C.

2. The amidase polypeptide of claim 1, wherein the conversion rate is at least 1.5 times that of the polypeptide of SEQ ID NO:2 at pH 9.5 at 25° C.

3. The amidase polypeptide of claim 1, wherein the conversion rate is at least 3 times that of the polypeptide of SEQ ID NO:2 at pH 9.5 at 25° C.

4. The amidase polypeptide of claim 1, wherein the amidase is capable of converting (R) 2-aminobutyramide to (R) 2-aminobutyrate in a racemic mixture of 2-aminobutyramide with reduced level of byproduct as compared to the polypeptide SEQ ID NO:2, as determined by fluorometry following derivatization with o-phthalaldehyde (OPA).

5. The amidase polypeptide of claim 4 in which the total byproduct is less than 7% of the total product determined by derivatization with OPA.

6. The amidase polypeptide of claim 4 in which the total byproduct is less than 5% of the total product determined by derivatization with OPA.

7. The amidase polypeptide claim 1 which comprises an amino acid sequence that is at least 80% identical to a reference sequence of SEQ ID NO: 18.

8. The amidase polypeptide of claim 1, wherein the amino acid sequence comprises one or more residue difference as compared to SEQ ID NO:2 at the following residues: X38; X149; X175; X278; X290; X291; X315; X317; X353; X363; X367; X376; X405; X414; X516; and X518.

9. The amidase polypeptide of claim 8, wherein the amino acid sequence includes at least one of the following features:
residue corresponding to X38 is a basic residue;
residue corresponding to X149 is a polar residue;
residue corresponding to X175 is a polar residue;
residue corresponding to X278 is a polar residue;
residue corresponding to X290 is a polar or acidic residue;
residue corresponding to X291 is a non-polar or aliphatic residue;
residue corresponding to X315 is an acidic or non-polar residue;
residue corresponding to X317 is an acidic residue;
residue corresponding to X353 is polar residue;
residue corresponding to X363 is a polar residue;
residue corresponding to X367 is an acidic residue;
residue corresponding to X376 is cysteine (C) or an aliphatic residue;
residue corresponding to X405 is an aliphatic residue residue corresponding to X414 is a polar residue;
residue corresponding to X516 is a constrained residue; and
residue corresponding to X518 is a polar residue.

10. The amidase polypeptide of claim 8, wherein the amino acid sequence includes at least one of the following features:
residue corresponding to X38 is arginine;
residue corresponding to X149 is threonine;
residue corresponding to X175 is serine;
residue corresponding to X278 is threonine or serine;
residue corresponding to X290 is serine, threonine, glutamine, or glutamic acid;
residue corresponding to X291 is methionine;
residue corresponding to X315 is glycine;
residue corresponding to X317 is aspartic acid;
residue corresponding to X353 is glutamine;
residue corresponding to X363 is serine;
residue corresponding to X367 is aspartic acid;
residue corresponding to X376 is isoleucine or cysteine;
residue corresponding to X405 is alanine;
residue corresponding to X414 is asparagine;
residue corresponding to X516 is proline; and
residue corresponding to X518 is threonine.

11. The amidase polypeptide of claim 10, wherein the amino acid sequence includes at least one of the following features:
residue corresponding to X290 is a polar or acidic residue;
residue corresponding to X291 is non-polar or aliphatic residue;
residue corresponding to X317 is an acidic residue;
residue corresponding to X367 is an acidic residue; and
residue corresponding to X414 is a polar residue.

12. The amidase polypeptide of claim 11, wherein the amidase amino acid sequence includes at least the following feature: residue corresponding to X290 is a polar or acidic residue.

13. The amidase polypeptide of claim 11, wherein the amino acid sequence includes at least the following feature: residue corresponding to X290 is serine, threonine, glutamine, or glutamic acid.

14. The amidase polypeptide of claim 11, wherein the amino acid sequence includes at least the following feature: residue corresponding to X291 is a non-polar residue.

15. The amidase polypeptide of claim 11, wherein the amino acid sequence includes at least the following features: residue corresponding to X291 is methionine.

16. The amidase polypeptide of claim 11, wherein the amino acid sequence includes at least the following features:
residue corresponding to X317 is an acidic residue;
residue corresponding to X367 is an acidic residue; and
residue corresponding to X414 is a polar residue.

17. The amidase polypeptide of claim 11, wherein the amino acid sequence includes at least the following features:
residue corresponding to X317 is aspartic acid;
residue corresponding to X367 is aspartic acid; and
residue corresponding to X414 is asparagine.

18. The amidase polypeptide of claim 11, wherein the amino acid sequence includes at least the following features:
residue corresponding to X290 is a polar or acidic residue;
residue corresponding to X317 is an acidic residue;
residue corresponding to X367 is an acidic residue; and
residue corresponding to X414 is a polar residue.

19. The amidase polypeptide of claim 11, wherein the amino acid sequence further includes one or more residue differences as compared to SEQ ID NO:2 at the following residues: X38; X149; X175; X278; X315; X317; X353; X363; X376; X405; X516; and X518.

20. The amidase polypeptide of claim 19, wherein the amino acid residue at the residue positions are selected from the following features:
residue corresponding to X38 is a basic residue;
residue corresponding to X149 is a polar residue;
residue corresponding to X175 is a polar residue;
residue corresponding to X278 is a polar residue;
residue corresponding to X315 is a non-polar residue;
residue corresponding to X353 is polar residue;
residue corresponding to X363 is a polar residue;
residue corresponding to X376 is cysteine (C) or an aliphatic residue;
residue corresponding to X405 is an aliphatic residue;
residue corresponding to X516 is a constrained residue; and
residue corresponding to X518 is a polar residue.

21. The amidase polypeptide of claim 20 in which the amino acid sequence further includes one or more of the following features:
residue corresponding to X38 is basic residue;
residue corresponding to X149 is a polar residue;
residue corresponding to X516 is a constrained residue; and
residue corresponding to X518 is a polar residue.

22. The amidase polypeptide of claim 21 in which the amino acid sequence further includes one or more of the following features:
residue corresponding to X175 is a polar residue;
residue corresponding to X278 is a polar residue;
residue corresponding to X315 is non-polar residue residue corresponding to X353 is polar residue;
residue corresponding to X363 is a polar residue;
residue corresponding to X376 is cysteine (C) or an aliphatic residue; and
residue corresponding to X405 is an aliphatic residue.

23. The amidase polypeptide of claim 20, wherein the amino acid sequence includes at least the following features:
residue corresponding to X290 is a polar or acidic residue;
residue corresponding to X315 is non-polar residue;
residue corresponding to X317 is an acidic residue;
residue corresponding to X367 is an acidic residue; and
residue corresponding to X414 is a polar residue.

24. The amidase polypeptide of claim 20, wherein the amino acid sequence includes at least the following features:
residue corresponding to X290 is serine, threonine, glutamine, or glutamic acid;
residue corresponding to X315 is glycine;
residue corresponding to X317 is aspartic acid;
residue corresponding to X367 is aspartic acid; and
residue corresponding to X414 is asparagine.

25. The amidase polypeptide of claim 20, wherein the amino acid sequence includes at least the following features:
residue corresponding to X38 is a basic residue;
residue corresponding to X149 is a polar residue;
residue corresponding to X290 is a polar or acidic residue;
residue corresponding to X317 is an acidic residue;
residue corresponding to X367 is an acidic residue; and
residue corresponding to X414 is a polar residue.

26. The amidase polypeptide of claim 19, wherein the amino acid sequence includes at least the following features:
residue corresponding to X38 is arginine;
residue corresponding to X149 is threonine;
residue corresponding to X290 is serine, threonine, glutamine, or glutamic acid;
residue corresponding to X317 is aspartic acid;
residue corresponding to X367 is aspartic acid; and
residue corresponding to X414 is asparagine.

27. The amidase polypeptide of claim 1, wherein the polypeptide is capable of stereospecifically converting (R)-2-aminobutyramide to-(R) aminobutyrate in a racemic mixture of 2-aminobutyramide to forms a stereomeric excess of at least 99% of (S)-2-aminobutyramide.

28. The amidase polypeptide of claim 27, wherein the amino acid sequence comprises a sequence selected from SEQ ID NO: 6, 8, 10, 12, 14, 16, 18, 20, and 22.

29. The amidase polypeptide of claim 1, wherein 1 g/L of the polypeptide is capable of converting at least 40% of 475 g/L of a racemic mixture of 2-aminobutyramide to (R)-2-aminobutyrate under reaction conditions of about pH 7.5 and about 35° C. in about 8 hrs.

30. The amidase polypeptide of claim 29 which is selected from SEQ ID NO: 6, 8, 10, 12, 14, 16, 18, 20, and 22.

31. The amidase polypeptide of claim 1, wherein 1 g/L of the polypeptide is capable of converting at least 40% of 300 g/L of a racemic mixture of 2-aminobutyramide to (R)-2-aminobutyrate under reaction conditions of about pH 7.5 and about 35° C. in about 20 hrs.

32. The amidase polypeptide of claim 31 which is selected from SEQ ID NO: 6, 8, 10, 12, 14, 16, 18, 20, and 22.

33. An engineered amidase polypeptide having an amino acid sequence selected from the group consisting of:
(a) an amino acid sequence that is at least 80% identical to SEQ ID NO: 2 wherein the amino acid sequence comprises one or more features selected from the group consisting of: residue corresponding to X38 is arginine; residue corresponding to X149 is threonine; residue corresponding to X175 is serine, residue corresponding to X278 is threonine or serine; residue corresponding to X290 is serine, threonine, glutamine, or glutamic acid; residue corresponding to X291 is methionine, residue corresponding to X315 is glycine; residue corresponding to X353 is glutamine; residue corresponding to X363 is serine; residue corresponding to X367 is aspartic acid; residue corresponding to X376 is isoleucine or cysteine; residue corresponding to X405 is asparagine; residue corresponding to X516 is proline; and residue corresponding to X518 is threonine, where position (X) refers to the corresponding position in SEQ ID NO 2; and
(b) an amino acid sequence that is encoded by a nucleic acid that hybridizes under high stringency conditions to the complement of SEQ ID NO: 1 (the polynucleotide sequence encoding SEQ ID NO: 2), wherein the encoded amino acid sequence comprises one or more features selected from the group consisting of: residue corresponding to X38 is arginine; residue corresponding to X149 is threonine; residue corresponding to X175 is serine, residue corresponding to X278 is threonine or serine; residue corresponding to X290 is serine, threonine, glutamine, or glutamic acid; residue corresponding to X291 is methionine, residue corresponding to X315 is glycine; residue corresponding to X353 is glutamine; residue corresponding to X363 is serine; residue corresponding to X367 is aspartic acid; residue corresponding to X376 is isoleucine or cysteine; residue corresponding to X405 is asparagine; residue corresponding to X516 is proline; and residue corresponding to X518 is threonine, where position (X) refers to the corresponding position in SEQ ID NO 2.

34. A polynucleotide encoding the engineered amidase polypeptide of claim 1.

35. The polynucleotide of claim 34 which is selected from SEQ ID NO: 5, 7, 9, 11, 13, 15, 17, 19, and 21.

36. An expression vector comprising the polynucleotide of claim 34 operably linked to a control sequence suitable for directing expression in a host cell.

37. The expression vector of claim 36, wherein the control sequence comprises a promoter.

38. The expression vector of claim 37, wherein the promoter comprises an E. coli promoter.

39. The expression vector of claim 36, wherein the control sequence comprises a secretion signal.

40. A host cell comprising the expression vector of claim 36.

41. The host cell of claim 40, which is E. coli.

42. A method of making an engineered amidase polypeptide, said method comprising:
(a) providing a host cell transformed with the expression vector of claim 36;
(b) culturing the transformed host cell in a culture medium under conditions that cause expression of an encoded amidase polypeptide; and
(c) optionally recovering the amidase polypeptide.

43. A method for resolving (S)-2-aminobutyramide from (R)-2-aminobutyramide, the method comprising contacting a mixture of (S)-2-aminobutyramide and (R)-2-aminobutyramide with the engineered amidase polypeptide of claim 1, under reaction conditions suitable to convert (R)-2-aminobutyramide to (R)-2-aminobutyrate, thereby forming (S)-2-aminobutyramide in stereometric excess.

44. The method of claim 43, wherein the mixture is a racemic mixture.

45. The method of claim 43, wherein the reaction conditions comprise a temperature of about 20 to about 40° C.

46. The method of claim 43, wherein the reaction conditions comprise a pH of about 6.5 to about 8.0.

47. The method of claim 43, wherein the reaction conditions comprise a pH of about pH 7.5.

48. The method of claim 43 wherein the reaction conditions comprise a pH of about 9.0 to about 10.0.

49. The method of claim 43, wherein the reaction conditions comprise a pH of about 9.5

50. The method of claim 43, wherein the reaction temperature is about 25° C.

51. The method of claim 43, wherein the (S)-2-aminobutyramide is formed in at least 99% stereomeric excess.

52. The method of claim 43, wherein the reaction conditions comprise a pH of about 7.5, a temperature of about 35° C., about 475 g/L of 2-aminobutyramide substrate, and about 1 g/L of engineered amidase polypeptide in a reaction time of about 8 hrs, wherein at least 40%, 45%, 46%, 47%, 48% or 49% of the racemic substrate is converted to (R)-2-aminobutyrate.

53. The method of claim 43, wherein the reaction conditions comprise a pH of about 9.5, a temperature of about 25° C., about 300 g/L of 2-aminobutyramide substrate in a reaction time of about 20 hrs, wherein at least 40%, 45%, 46%, 47%, 48% or 49% of racemic substrate is converted to (R) 2-aminobutyrate.

54. A method for resolving (S)-2-aminobutyramide from (R)-2-aminobutyramide, the method comprising contacting a lower carboxylic acid salt or sulfuric acid salt of 2-aminobutyramide with an amidase polypeptide under reaction conditions suitable to convert (R)-2-aminobutyramide to (R)-2-aminobutyrate, thereby forming (S)-2-aminobutyramide in stereomeric excess of (R)-2-aminobutyramide.

55. The method of claim 54, wherein the lower carboxylic acid or sulfuric acid salt of 2-aminobutyramide is a formate, acetate, sulfate, or propionate salt.

56. The method of claim 55, wherein the lower carboxylic acid salt is the acetate salt of 2-aminobutyramide.

57. The method of claim 54, wherein the (S)-2-aminobutyramide is formed in at least 99% stereomeric excess.

58. The method of claim 54 in which the amidase polypeptide comprises an amino acid sequence selected from the group consisting SEQ ID NO: 2, 23, 24, 25, 26, 27, 28, 29, 30, 31, 32, 33, 34 and 35.

59. The method of claim 54 in which the amidase polypeptide comprises an engineered amidase polypeptide of claim 1.

60. In a method for synthesis of (2S)-2-(2-oxopyrrolidin-1-yl)butanamide, a step in the method comprising contacting a mixture of (R)-2-aminobutyramide and (S)-2-aminobutyramide with an engineered amidase polypeptide of claim 1 to form (S)-2-aminobutyramide in stereomeric excess.

61. The method of claim 60, wherein the (S)-2-aminobutyramide is formed in at least 99% stereomeric excess.

62. The method of claim 61 further comprising isolating the (S)-2-aminobutyramide.

63. The method of claim 62, wherein the (S)-2-aminobutyramide is isolated by extraction with a polar organic solvent.

64. The method of claim 63, wherein the polar organic solvent is selected from acetonitrile, methanol, ethanol, butanol, isopropanol, isobutanol, tert-butanol, acetone, and methylethylketone.

65. The method of claim 63, wherein the extraction is selected from a step-wise and continuous extraction.

66. The method of claim 63, wherein the extraction is a step-wise extraction with isopropanol.

67. In a method for synthesis of (2S)-2-(2-oxopyrrolidin-1-yl)butanamide, a step in the method comprising contacting a lower carboxylic acid salt or sulfuric acid salt of a mixture of (R)-2-aminobutyramide and (S)-2-aminobutyramide with an amidase polypeptide to form (S)-2-aminobutyramide in stereomeric excess of (R)-2-aminobutyramide.

68. The method of claim 67, wherein the lower carboxylic acid salt or sulfuric acid salt of 2-aminobutyramide is a formate, acetate, sulfate, or propionate salt.

69. The method of claim 68, wherein the lower carboxylic acid salt of 2-aminobutyramide is the acetate salt.

70. The method of claim 67 further comprising isolating the (S)-2-aminobutyramide.

71. The method of claim 70, wherein the (S)-2-aminobutyramide is isolated by extraction with a polar organic solvent.

72. The method of claim 71, wherein the polar organic solvent is selected from acetonitrile, methanol, ethanol, butanol, isopropanol, isobutanol, tert-butanol, acetone, and methylethylketone.

73. The method of claim 71, wherein the extraction is selected from a step-wise and continuous extraction.

74. The method of claim 73, wherein the extraction is a step-wise extraction with isopropanol.

75. The method of claim 67, wherein the amidase polypeptide comprises an amino acid sequence selected from the group consisting of SEQ ID NO: 2, 23, 24, 25, 26, 27, 28, 29, 30, 31, 32, 33, 34 and 35.

76. The method of claim 67, wherein the amidase polypeptide comprises an engineered amidase polypeptide of claim 1.

77. A composition comprising a lower carboxylic acid salt or sulfuric acid salt of a compound of structural formula (I), formula (III), or mixtures thereof: 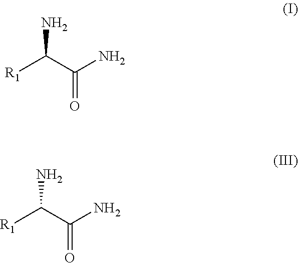
wherein R₁ is a lower (C₁-C₄) alkyl.

78. The composition of claim 77, wherein the compound of structural formula I is (S)-2-aminobutryamide and the compound of structural formula (II) is (R)-2-aminobutyramide.

79. The composition of claim 78, wherein the lower carboxylic acid salt or sulfuric acid salt of 2-aminobutyramide is selected from formate, acetate, sulfate and propionate salt.

80. The composition of claim 79, wherein the lower carboxylic acid salt of 2-aminobutyramide is the acetate salt of (R)-2-aminobutyramide or (S)-2-aminobutyramide, or mixtures thereof.

81. The composition of claim 80, comprising a racemic mixture of the acetate salt of 2-aminobutyramide.

82. The composition of claim 77, further comprising an amidase polypeptide.

83. The composition of claim 82, wherein the amidase polypeptide comprises an engineered amidase polypeptide capable of stereospecifically converting (R)-2-aminobutyramide to (R) 2-aminobutyric acid with an increased conversion rate as compared to the polypeptide of SEQ ID NO: 2 at pH 7.5 at 35° C.

84. The composition of claim 82, wherein the amidase polypeptide comprises an amino acid sequence selected from the group consisting of SEQ ID NO:2, 23, 24, 25, 26, 27, 28, 29, 30, 31, 32, 33, 34 and 35.


SEQUENCE LISTING (35 sequences)

SEQ ID NO: 1 - DNA, 1560 bp, Artificial Sequence
  Codon optimized O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgccctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtggacattctgtctgaacgcatttttgctccggcgggtatgaagcgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggccacggcggcgccctgcgcggctggcgctgccaacgttggcactgtgctgacgaacgcctgagtacgatcgcgatgttcaacttcgaaggtggtgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacggtcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgagcatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccg

SEQ ID NO: 2 - protein, 520 aa, Ochrobactrum anthropi
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGALRGWRCQRWHCADERLSTIAMFNFEGGASEVAFKLMNIALGVSSSEVSRVEADSAWFGSWLDDETGLVLSLEDAGHGRMKARFGTSPEMMDVVSANEARSAVTTIRRDGETIELVRASENLRLSMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 3 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggtgccctgcgcggctggcgctgccaacgttggcactgtgctgacgaacgcctgagtacgatcgcgatgttcaacttcggaggtggtgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacggtcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgagcatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 4 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGALRGWRCQRWHCADERLSTIAMFNFGGGASEVAFKLMNIALGVSSSEVSRVEADSAWFGSWLDDETGLVLSLEDAGHGRMKARFGTSPEMMDVVSANEARSAVTTIRRDGETIELVRASENLRLSMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 5 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccgggttcccactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggtgccatgcgcggctggcgctgccaacgttggcactgtgctgacgaacgcctgagtacgatcgcgatgttcaacttcggaggtggtgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacggtcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgagcatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 6 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGAMRGWRCQRWHCADERLSTIAMFNFGGGASEVAFKLMNIALGVSSSEVSRVEADSAWFGSWLDDETGLVLSLEDAGHGRMKARFGTSPEMMDVVSANEARSAVTTIRRDGETIELVRASENLRLSMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 7 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggtgccctgcgcggctggcgctgccaacgttggcactgtgctgacgaacgcctgagtacgatcgcgatgttcaacttcggaggtgatgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacgatcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgaacatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 8 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGALRGWRCQRWHCADERLSTIAMFNFGGDASEVAFKLMNIALGVSSSEVSRVEADSAWFGSWLDDETGLVLSLEDAGHDRMKARFGTSPEMMDVVSANEARSAVTTIRRDGETIELVRASENLRLNMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 9 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggtactctgcgcggctggcgctgccaacgttggcactgtgctgacgaacgcctgagtacgatcgcgatgttcaacttcggaggtgatgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacgatcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgaacatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 10 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGTLRGWRCQRWHCADERLSTIAMFNFGGDASEVAFKLMNIALGVSSSEVSRVEADSAWFGSWLDDETGLVLSLEDAGHDRMKARFGTSPEMMDVVSANEARSAVTTIRRDGETIELVRASENLRLNMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 11 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggtcagctgcgcggctggcgctgccaacgttggcactgtgctgacgaacgcctgagtacgatcgcgatgttcaacttcggaggtgatgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacgatcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgaacatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 12 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGQLRGWRCQRWHCADERLSTIAMFNFGGDASEVAFKLMNIALGVSSSEVSRVEADSAWFGSWLDDETGLVLSLEDAGHDRMKARFGTSPEMMDVVSANEARSAVTTIRRDGETIELVRASENLRLNMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 13 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggtgagctgcgcggctggcgctgccaacgttggcactgtgctgacgaacgcctgagtacgatcgcgatgttcaacttcggaggtgatgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacgatcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgaacatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 14 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGELRGWRCQRWHCADERLSTIAMFNFGGDASEVAFKLMNIALGVSSSEVSRVEADSAWFGSWLDDETGLVLSLEDAGHDRMKARFGTSPEMMDVVSANEARSAVTTIRRDGETIELVRASENLRLNMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 15 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggttcgctgcgcggctggcgctgccaacgttggcactgtgctgacgaacgcctgagtacgatcgcgatgttcaacttcggaggtgatgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacgatcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgaacatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 16 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGSLRGWRCQRWHCADERLSTIAMFNFGGDASEVAFKLMNIALGVSSSEVSRVEADSAWFGSWLDDETGLVLSLEDAGHDRMKARFGTSPEMMDVVSANEARSAVTTIRRDGETIELVRASENLRLNMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 17 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggtactctgcgcggctggcgctgccaacgttggcactgtgctgacgaacgcctgagtacgatcgcgatgttcaacttcggaggtgatgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacgatcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgaacatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcctcgcacgcagccgtga

SEQ ID NO: 18 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGTLRGWRCQRWHCADERLSTIAMFNFGGDASEVAFKLMNIALGVSSSEVSRVEADSAWFGSWLDDETGLVLSLEDAGHDRMKARFGTSPEMMDVVSANEARSAVTTIRRDGETIELVRASENLRLNMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYPRTQP

SEQ ID NO: 19 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcgtcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggtacgcactactcctattgcaacggtaactttcgtattctggcggacctgatcgaagcgcatactggccgtaccctggtggacattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatgagactggtggtaagcgcttaaccggtcacggcggttcgctgcgcggctggcgctgccaacgttggcactgtgctgacgaacgcctgagtacgatcgcgatgttcaacttcggaggtgatgcaagtgaggtcgcgtttaaattgatgaatattgccttgggcgtgtccagtagcgaagtttcccgtgtcgaagcagacagcgcatggttcggctcatggttagatgatgagactggtctggttttatccctggaagatgcaggtcacgatcgtatgaaagcgcgctttggtacgagtccggaaatgatggatgtagtctcggcaaatgaagctcgcagcgcagtgaccactattcgccgcgacggtgagaccattgaattagtacgtgcttccgagaacctgcgtctgaacatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 20 - protein, 520 aa, Artificial Sequence
  variant of O. anthropi DAP
MSKFDTSALEAFVRHIPQNYKGPGGVVAVVKDGEVVLRHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGTHYSYCNGNFRILADLIEAHTGRTLVDILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHETGGKRLTGHGGSLRGWRCQRWHCADERLSTIAMFNFGGDASEVAFKLMNIALGVSSSEVSRVEADSAWFGSWLDDETGLVLSLEDAGHDRMKARFGTSPEMMDVVSANEARSAVTTIRRDGETIELVRASENLRLNMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 21 - DNA, 1563 bp, Artificial Sequence
  variant of O. anthropi DAP
atgtcgaaattcgacaccagcgcgctggaagcctttgtgcgtcatattccgcagaattataaaggtcctggcggtgttgtggcagtcgtaaaagatggggaagtagttctgcagcacgcttggggcttcgcggacttacgcacgcgtacaccgatgactctggatacccgcatgccaatctgctctgtctccaagcaatttacctgtgcggtcctgctggatgcggtgggcgaaccggagctgctggatgatgctctggaagcttacttggataaattcgaagatgaacgtccggcagtgcgtgacttgtgtaacaatcagtcgggcctgcgcgactattgggcgttgtcggttctgtgcggcgcggatccggagggcgtgttcttgccagcacaggcgcagtcattgctgcgccgcctgaaaaccactcattttgagccaggttcccactactcctattgcaacggtaactttcgtattctggcggacctgatcgaagcgcatactggccgtaccctggtgagcattctgtctgaacgcatttttgctccggcgggtatgaaacgtgccgaactgatcagtgacaccgctttgttcgacgaatgtaccggttatgaaggtgataccgtgcgtggtttcctgccggccaccaaccgtatccagtggatgggggacgcgggtatctgcgcttcactgaacgatatgattgcgtgggaacagtttattgatgcgacccgtgatgatgaatctggcctgtaccgccgtctgagtggcccgcagaccttcaaggatggcgtggccgctccgtacggcttcgggctgaacctgcatagcactggtggtaagcgcttaaccggtcacggcggtgccctgcgcggctggcgctgccaacgttggcactgtgctgacgaacgcctgagtacgatcgcgatgttcaacttcggaggtggtgcaagtgaggtcgcgtttaaattgatgaatattgccttgggcgtgtccagtagcgaagtttcccgtgtcgaagcagacagcgcatggttcggctcatggttagatcaagagactggtctggttttatccctggaaagcgcaggtcacggtcgtatgaaagcgcgctttggtacgtgtccggaaatgatggatgtagtctcggcaaatgaagctcgcagcgcagtgaccactattcgccgcgacggtgagaccattgaattagcacgtgcttccgagaacctgcgtctgagcatgaaacgtgttaaaggggaagctaaacatgacattatcggtcgctaccattccgatgaattagatgcagacctgctgctggtctcggaagggggggccatttatggtgcttttgaaggctttttggggaaatctgacatgtatccgctgtactcagttggtagcgacgtctggttactgccggtgcaacgttctatggacgcgccatctccgggtgaatggaaattagtgttccgtcgtgacgataagggcgaaatcaccggtctgtctgttggctgctggctggcacgtggtgttgaatatcgtcgcgtgcagccgtga

SEQ ID NO: 22 - protein, 520 aa, Artificial Sequence
  variant of O. anthropi DAP
MSKFDTSALEAFVRHIPQNYKGPGGVVAVVKDGEVVLQHAWGFADLRTRTPMTLDTRMPICSVSKQFTCAVLLDAVGEPELLDDALEAYLDKFEDERPAVRDLCNNQSGLRDYWALSVLCGADPEGVFLPAQAQSLLRRLKTTHFEPGSHYSYCNGNFRILADLIEAHTGRTLVSILSERIFAPAGMKRAELISDTALFDECTGYEGDTVRGFLPATNRIQWMGDAGICASLNDMIAWEQFIDATRDDESGLYRRLSGPQTFKDGVAAPYGFGLNLHSTGGKRLTGHGGALRGWRCQRWHCADERLSTIAMFNFGGGASEVAFKLMNIALGVSSSEVSRVEADSAWFGSWLDQETGLVLSLESAGHGRMKARFGTCPEMMDVVSANEARSAVTTIRRDGETIELARASENLRLSMKRVKGEAKHDIIGRYHSDELDADLLLVSEGGAIYGAFEGFLGKSDMYPLYSVGSDVWLLPVQRSMDAPSPGEWKLVFRRDDKGEITGLSVGCWLARGVEYRRVQP

SEQ ID NO: 23 - protein, 520 aa, Ochrobactrum anthropi; identical to SEQ ID NO: 2

SEQ ID NO: 24 - protein, 520 aa, Ochrobactrum anthropi
MSKFDLSALETFVRTIPQHYKGPGGAVAVLKDGKVVLRHAWGFADLSTRKAMTPETRMPICSVSKQFTCAVLLDSVGEPEVLDDALAAYLDKFAEKRPSVRDLCNNQSGLRDYWALTVLCGADPEGVFLPEQAQSLLRRLKTTHFAPGTHYSYCNGNFRILADLIEQHTGRSLIELLSERIFQPAGMKTAELIPDTALFDECTGYEGDTVRGFLPAVNRIHWMGDAGICASLDDMIAWEQFIDATRDDESGIYRRLSGLQTFSDGAAAPYGLGLKFEETGGKRLTGHGGALRGWRCQRWHCADERLSTIAMFNFEGGASDVAFKLMNIALGVPTSEQVRVAADAVWFGSWLDHETGLVLSLEDAGRGRMKARFGTGPEMMDVVGENEARSSMTAIRRDGDTIHLAREDENLSLTMQRLKGAAKQDIAGHYRSDELEADLLIVDEGGAFYGAFEGFLGKSDMYSLYEAGPDVWLLPVQRSMDAPSPGEWKLVFHRDAKGEITGMTVGCWLARHVDYRRIQE

SEQ ID NO: 25 - protein, 518 aa, Brucella suis
MPNIDLPTLEAFVHAIPQNYKGPGGAVAVVRNGEIVLRHAWGFADLAARKAMTPETRMPICSVSKQFTCAVLLDCIGEPEMLDSALAAYLDQFEDGRPAVRDLCNNQSGLRDYWALTVLCGAAPEGIFLPDQAQNLLRRLKTTHFAPGTHYSYCNGNFRILADLIEQHTGRSLADLLAERIFAPAAMKTAELIPDTALFNECTGYEGDTVRGFLPAINRIHWLGDAGICASLDDMIAWEQFIDRTRHDENGLYRRLSSPQTFADGAPAPYGFGLKFEETGGKRLTGHGGALRGWRCQRWHCADERISTIVMFNFEGNASDAALKMMNAALGIPPAKPVRAQANPGWFGSWLNPETGLVLSLEDAGGGRMKARFGTGPEIMDISGENEAQSSMTTLRRDGDMIHLARKDENLHLAMHRLKGEARQDIAGRYRSDELEADLLLVSEGGAIYGAFEGFLGKSDMYPLYAAGPDVWLLPVQRSMDAPSPGEWKLVFHRDAAGRITGVTVGCWLARGVEYKRL

SEQ ID NO: 26 - protein, 518 aa, Brucella melitensis
MPNIDLPTLEAFVHAIPQNYKGPGGAVAVVRNGEIVLRHAWGFADLAARKAMTPETRMPICSVSKQFTCAVLLDCIGEPEMLDSALAAYLDQFEDGRPAVRDLCNNQSGLRDYWALTVLCGAAPEGIFLPDQAQNLLRRLKTTHFAPGTHYSYCNGNFRILADLIEQHTGRSLADLLAERIFAPAAMKTAELIPDTALFNECTGYEGDTVRGFLPAINRIHWLGDAGICASLDDMIAWEQFIDRTRHDENGLYRRLSSPQTFADGAPAPYGFGLKFEETGGKRLTGHGGALRGWRCQRWHCADERISTIVMFNFEGNASDAALKMMNAALGIPPAKPVRAQANPGWFGSWLNPETGLVLSLEDAGGGRMKARFGTGPEKMDISGENEAQSSMTTLRRDGDMIHLARKDENLHLAMHRLKGEARQDIAGRYRSDELEADLLLVSEGGAIYGAFEGFLGKSDMYPLYAAGPDVWLLPVQRSMDAPSPGEWKLVFHRDAAGRITGVTVGCWLARGVEYKRL

SEQ ID NO: 27 - protein, 518 aa, Brucella ovis
MPNIDLPTLEAFVHAIPQNYKGPGGAVAVVRNGEIVLRHAWGFADLAARKAMTPETRMPICSVSKQFTCAVLLDCIGEPEMLDSALAAYLDQFGDGRPAVRDLCNNQSGLRDYWALTVLCGAAPEGIFLPDQAQNLLRRLKTTHFAPGTHYSYCNGNFRILADLIEQHTGRSLADLLAERIFAPAAMKTAELIPDTALFNECTGYEGDTVHGFLPAINRIHWLGDAGICASLDDMIAWEQFIDRTRHDENGLYRRLSSPQTFADGAPAPYGFGLKFEETGGKRLTGHGGALRGWRCQRWHCADERISTIVMFNFEGNASDAALKMMNAALGIPPAKPVRAQANPGWFGSWLNPETGLVLSLEDAGGGRMKARFGTGPEIMDISGENEAQSSMTTLRRDGDMIHLARKDENLHLAMHRLKGEARQDIAGRYRSDELEADLLLVSEGGAIYGAFEGFLGKSDMYPLYAAGPDVWLLPVQRSMDAPSPGEWKLVFHRDAAGRITGVTVGCWLARGVEYKRL

SEQ ID NO: 28 - protein, 516 aa, Rhodobacter sphaeroides
MTLDLDALDRALDALPNLFRGPGGVAGVVKDGQVVASRAWGYADLTRRRPMETGTRLPICSISKQFTCGVLLDTLGDTAAYDARVAEFLPQFEGPLPTLRQLCDNQSGLRDYWALTVLQGAEATQTFRREDALPLIARMKTGHFPPGTAYSYCNCNFRIVSEILESETGRALPDLYAERIFGPAGMRTAELTSDTRHPADEVVGYEGSDAVGFFPADNGIFWIGDAGISASLQDMLAYESWIDATRDDENSIYRRISVPPAYVCGTPASYGFGLSHETVAGLKVTGHGGALRGFRAQRFHAADERLSVVVIFNHEASAHAAASSLLAAALGHEAPKGAGPEGWAGQWLDPESGLLLRVGEDAEGLTLRFATGPDRLTVGEDGVPRGAGVSLAREGATLVMNRTSDNLTVRAEPLPVVAVADAGEIAGRYHARELEADLVIEARDGGAYAGFEGLLGAGPMERLHPVGPDVWIVTTRRSMDAPAPGDWTLQVRREGGAVAGLRLGCWLARRIDYARV

SEQ ID NO: 29 - protein, 516 aa, Rhodobacter sphaeroides
MTLDLDALDRALDALPNLFRGPGGVAGVVKDGQVVASRAWGYADLTRRRPMETGTRLPICSISKQFTCGALLDTLGDTAAYDARVAEFLPQFEGPLPTLRQLCDNQSGLRDYWALTVLQGAEAAQTFRREDALPLIARMKTGHFPPGTAYSYCNCNFRIVSEILESETGRALRDLYAERIFGPAGMRTAELTSDTRHPADEVVGYEGSDAVGFFPADNGIFWIGDAGISASLQDMLAYESWIDATRNDENSIYRRISVPPAYVCGTPASYGFGLSHETVAGVKVTGHGGALRGFRAQRFHAADERLSVVVIFNHEASAHAAASSLLAAALGHEAPKGARPEGWAGQWLDPESGLLLRVGEDAEGLTLRFATGPDRLTAGEDGVPRGAGVSLAREGAMLVMNRTSDNLTVRAEPLPVVAVADAGEIAGRYHARELEADLVIEARDGGAYAGFEGLLGAGPMERLHPVGPDVWIVTTRRSMDAPAPGDWTLQVRREGGAVTGLRLGCWLARRIDYARV

SEQ ID NO: 30 - protein, 494 aa, Aspergillus oryzae
MQNLVPMTTSTLMPICSITKQMVCMILKDLERNPTPEMVKRGNVSRQFSDALHQMLHPDLTTNTALELEHLCHNQSGIRDYWAMSMFWGAHPDGVFRLEEDAKKALERTKSLHFEPGTQYSYCNLNFYILARLIENVSGQTLSALLTERLFLPAQMKTARLCADNANLPPPCVGYEGTESSGFIPAINRMQWSGDAGVVASLKDMIAYEHYLLTCWDEEKSVYRAIAQQQSFKDGTVAQYGYGLKHVTIRGMATIGHGGALRGFRLHRIQAPSEKVAVVVMLNHQADAEEAAQDIMKSVLDIFPSHVPQSDPVNPSPDWFGTFYDPDAQLVVEIRHGGQGHVIVSYAGSDETLICVGENEAGSNNTVGIIDGGIPKLRRLVDNRVLDVKRVATGYPPHKDDYLGEYYCAEIDSTFRCSGAGGMLYGHFEGYLGQGPPHLMRYIGEDIWMLSCPRGLDAPAPGNWTVVFQRADHGDITSVTIGCWLARKVVFVKR

SEQ ID NO: 31 - protein, 525 aa, Gluconobacter oxydans
MSDSFSARLEAAVSALPARFPGPGGAVAVLKDGEVLVRHGWGYANVERRIPFTPSTLFRMCSITKQFTCGTLLDLYDDPSELDADVDARLPQLDEPSPGMLHLAHNQSGLRDYWAVAMLHGAPIEGYFGDREARRVIDGTRTLQFQPGTSYSYVNQNFRLISDILQDRTGRSFAELLQTSIFNPVGMERAILAAETRAMPDGTVGYEGSVESGFRPAINNIWWTGDAGLGASLDDMIAWERFIDETRDAPDSLYRRLTVPVTFSDGQPAPYGFGLQRTKMFGRDVTMHGGALRGWRSHRLHVASERLSVVVMFNHMSAAQVASAQILAAALGVPYEPERSTQQPTALYGTYLERETGLSARIEPAPGGAKLRYLMVPELLEGISATRAEAGSVVVKAQETAEGAEAVMERPGENRTSILARCDETPGEDIAELAGVYRCEELDEAEVTIELAGGVVYGGFSGILGDGRMEMLQRLAKDVWVLPCPRALDHTAPGDWTLAFERQGGSVTAVRVGCWLARDLMYQRV

SEQ ID NO: 32 - protein, 520 aa, Roseovarius sp.
MTAIDLAALETTLDALPKAFPGPGGLVGVVKDGKIVASRAWGYADLDAGAPLTAATRMPICSISKQFTCGVLLAALGEPEQYDARVAEFLPRYEGPMPTLRQLCDNQSGLRDYWALTVLQGGLAEQTFAREHALPLLARMKTGHFTPGSEYSYCNCNFRIISEIIESETGQALPDLYRRHIWEPAGMTTAVLTSDTRQPEDGVIGYEGSDATGFFPADNGIFWIGDAGISASLDDMLAYEAWIDAEREDPQSLYRRLSEPPRFVDGAPAQYGYGLAHETIGGVAFTGHAGALRGFRAHRLNARSERLSVVVLFNHEADAHGAAEMVARAAMGTPASEPTFIAEEWDGLWLDEARGLTATLKTGRRAVELGYAVGPQPTEAGQDGALAAAGVRITRDGAGLAMARPGENTIRALTPLVPIDTADGREIAGRYRSEELEAEMQIEARDGGVYARFDGLLGTGRFERMAPLARDVWRLATRRAMDAPAPGDWTLIVTRREDGSVSGARLGCWLARQIPYEKTA

SEQ ID NO: 33 - protein, 536 aa, Gibberella zeae
MPVSKQIVEDILKTIPSRIRGPGGAIAVLSDSALVDHRVWGYADFNRRIPMTPEVQIPICSISKQMLCALVVDLERNPTPALKAKGDVKEQFETKLKELLPELAKTGKLKLDDLCNNRSGIRDYWAITLLWGAKPEDRYSLADHNGPMLDSLKSFHFEPGTEYSYSNTNFNIVGRVVEACTGESLASLLNERIFKPAGMKTAELCADSAKLPPPCVGYEGDELRGYIPAENRIEWAGDAGIIASLTDMIAYESFLDRSVFDPESWYRKIFQPSTFSDGVPSTYAQGLAHTEIGSVGAIGHGGALRGYRLYRAHVPEERLSVVVMFNHEADAATLSEYIIQNILGLQKPMAPALVDASPTWPGVYLDEGTQLVITVTLGQKGQVLVTYANYVEPVKLTDPERGESKGMAAVINGDILRIQRLKENRAITAKRLTLGTIAIDSSPIEGDFYSPEINSTFHCVNQGGLVFGTFDGFLGQSPAQFMRYLGDDVWALANPRGMDAKPPGDWTVVFRRDDKGTVVGLTIGCWLARKIEYVRK

SEQ ID NO: 34 - protein, 531 aa, Aspergillus niger
MQAILEAVPSRYRGPGGAVAIIKDGELAGKLTWGYADLRKRISMTSSTLMPICSITKQMLCLILKDLERNPTPAMVERGNIPQQLSDAFRQLVSPELIDSNGLRIDHLCNNQSGIRDYWAMSMFWGTQPEGEFSLAGDAKKALDRTKSLHFQPGTQFSYCNLNFHILARIVEQVSGASLGKLLSERLFLPAGMKTAFLCADNAELPAPCVGYEGNESFGYIPAVNRTQWSGDAGVVASLEDMIAYECYLDQAWADRGSVYREIAKEPSFDDGTPAAYGYGLEHIMFGQTTTIGHGGAIRGYRLHRTQVPSERLAVVVMLNHEIDAAEVAGHILQEAHSLPAQHGLLSMPVANWVGFYFDPDAQLVVEVRLGGPGEVILTYTGDEETLKLTERCEARSDLATATLCSDTLLLHRKYDNRHIQAKRITAGQQPTKGDYVGQYHCAEVDSIFQCSGDGGMLYGSFKGFLGHGPAELMRYLGDDIWFLACARGLDAPAPGNWTVVFQRDVQGEVVGVTFGCWLARKVTFVKDRTP

SEQ ID NO: 35 - protein, 597 aa, Aspergillus niger
MPVWVKGAVRPEPAVSRTGARLPSGSSLKKQALDLNEYLYGYCFFSPSCTEIIFTLNLAFQVTFPNMQAILEAVPSRYRGPGGAVAIIKDGELAGKLTWGYADLRKRISMTSSTLMPICSITKQMLCLILKDLERNPTPAMVERGNIPQQLSDAFRQLVSPELIDSNGLRIDHLCNNQSGIRDYWAMSMFWGTQPEGEFSLAGDAKKALDRTKSLHFQPGTQFSYCNLNFHILARIVEQVSGASLGKLLSERLFLPAGMKTAFLCADNAELPAPCVGYEGNESFGYIPAVNRTQWSGDAGVVASLEDMIAYECYLDQAWADRGSVYREIAKEPSFDDGTPAAYGYGLEHIMFGQTTTIGHGGAIRGYRLHRTQVPSERLAVVVMLNHEIDAAEVAGHILQEAHSLPAQHGLLSMPVANWVGFYFDPDAQLVVEVRLGGPGEVILTYTGDEETLKLTERCEARSDLATATLCSDTLLLHRKYDNRHIQAKRITAGQQPTKGDYVGQYHCAEVDSIFQCSGDGGMLYGSFKGFLGHGPAELMRYLGDDIWFLACARGLDAPAPGNWTVVFQRDVQGEVVGVTFGCWLARKVTFVKDRTP